(19) United States
(12) Patent Application Publication
(10) Pub. No.: US 2024/0068049 A1
(43) Pub. Date: Feb. 29, 2024

(54) ASSAYS, KITS AND METHODS FOR DETECTION OF BOVINE RESPIRATORY DISEASE COMPLEX-ASSOCIATED PATHOGENS

(71) Applicant: PURDUE RESEARCH FOUNDATION, West Lafayette, IN (US)

(72) Inventors: Mohit VERMA, West Lafayette, IN (US); Suraj MOHAN, San Jose, CA (US); Ana PASCUAL-GARRIGOS, West Lafayette, IN (US); Haley BROUWER, Naples, FL (US); Jennifer KOZIOL, Okmulgee, OK (US); Jon SCHOONMAKER, West Lafayette, IN (US); Timothy JOHNSON, West Lafayette, IN (US)

(21) Appl. No.: 18/252,711

(22) PCT Filed: Nov. 11, 2021
(86) PCT No.: PCT/US2021/059032

§ 371 Date: May 11, 2023

Related U.S. Application Data
(60) Provisional application No. 63/170,771, filed on Apr. 5, 2021.
(60) Provisional application No. 63/112,412, filed on Nov. 11, 2020.

(51) Int. Cl. C12Q 1/689

(52) U.S. Cl. CPC C12Q 1/689

(57) ABSTRACT

Assays, kits, and methods that target and/or detect the presence of bovine respiratory complex (BRD)-associated pathogens in a sample. These assays, kits and methods can be portable and capable of providing fast (within 45 minutes) and accurate (at least 96%) results in the field, eliminating the need for a laboratory and other complex equipment.

### PRIORITY

This application is related to, and claims the priority benefit of, and is a 35 U.S.C. 371 national stage application of International Patent Application No. PCT/US2021/059032 filed Nov. 11, 2021, which is related to and claims the priority benefit of: (a) U.S. Provisional Patent Application No. 63/112,412 filed Nov. 11, 2020 to Verma et al.; and (b) U.S. Provisional Patent Application No. 63/170,771 filed Apr. 5, 2021 to Verma et al. The contents of the aforementioned applications are hereby incorporated in their entireties into this disclosure.

### STATEMENT REGARDING FEDERALLY SPONSORED RESEARCH

This invention was made with government support under Grant No. 2020-68014-31302 awarded by the National Institute of Food and Agriculture, U.S. Department of Agriculture. The United States Government has certain rights in the invention.

### SEQUENCE LISTINGS

The sequences herein (at least in Tables 2 and 3) are also provided in computer readable form encoded in a file filed herewith and incorporated herein by reference. The information recorded in computer readable form is identical to the written Sequence Listings provided below, pursuant to 37 C.F.R. § 1.821(f).

### BACKGROUND

Bovine respiratory disease complex (BRD) is a general term for respiratory disease in cattle and is a major cause of economic losses. Indeed, BRD is the most common and costliest disease affecting North American feedlot cattle, veal calves, weaned dairy heifers, and beef calves, with an approximate incidence rate of 15%.

BRD serves as an umbrella term for a series of respiratory illnesses caused by infections occurring along the respiratory tract. Cattle afflicted with BRD are likely to develop pneumonia with physical symptoms including elevated temperatures, nasal discharge, depression, and reduced appetite. While BRD is treatable, unmanaged cases of BRD can lead to expensive diagnosis/treatments, high morbidity rates, and decreases in overall meat quality. Many infectious agents have been associated with BRD, including infections in combination with viruses (such as bovine respiratory syncytial virus or parainfluenza virus) and bacteria (with the most common including Pasteurella multocida, Mannheimia haemolytica, Histophilus somni, and Mycoplasma bovis). Economic losses to the North American feedlot industry have been reported to be as high as $900 million dollars annually, due to losses in production, increased labor expenses, pharmaceutical costs, and death.

As the clinical symptoms associated with BRD can be non-specific, subtle, and exhibit a rapid onset, fast and accurate diagnosis of BRD presents a significant challenge to the North American cattle industry. Medical treatment (e.g., prescription of antibiotics) varies depending on the implicated pathogen and, therefore, it is important to diagnose at the earliest stage possible. Often, however, cattle with BRD are detected late in the disease process or not at all.

Conventional methods for detecting and/or diagnosing pathogenic causes of BRD include observation of physical symptoms in cattle, such as loss of appetite, elevated temperature, and depression, and performing laboratory assays on collected biological materials (e.g., serology, cell culture, immunohistochemistry, and in-situ hybridization). These current detection methods suffer drawbacks that make effective diagnosis and treatment of BRD difficult.

Physical indicators do not determine the causative pathogen and thus, are not sufficient for guiding appropriate therapy. Additionally, assessing the physical symptoms of cattle to determine a BRD disease state often results in inconsistent findings.

In an attempt to address these issues, there have been a growing number of diagnostic tests developed to detect BRD pathogens. While some test assays have had nearly three decades of development and standardization, they still suffer from significant disadvantages. Conventional diagnostics cannot be performed in the field and, thus, require additional time and money to ship the samples from farms to laboratories for processing. As such, laboratory-based tests require long processing times, especially when a large number of livestock samples are submitted for testing. This testing delay, or bottleneck between testing and diagnosis, can result in long wait times for feedlot operators or veterinarians and loss of valuable livestock.

Further, conventional diagnostic tests require isolation by culturing, special technical skills, and specialized equipment. Culturing for the detection of a BRD pathogen in clinical material is often time-consuming and the sensitivity of conventional methods is low. Traditional culture methods are time-consuming, require several days to obtain bacterial isolates, and some species such as M. bovis and H. somni grow poorly—a characteristic that may result in an under representation of the role of these pathogens in BRD.

Polymerase chain reaction (PCR) assays offer an alternative approach for detecting the presence of BRD in samples through amplification of targeted DNA sequences unique to pathogenic strains. However, for much of the same reasons as the previously described, these PCR assays are restricted to the laboratory setting and are not available in the field.

A possible solution to the equipment limitation is Recombinase Polymerase Amplification (RPA) to detect the presence of BRD pathogens in deep nasopharyngeal swabs. This technique allows for the binding and amplification of target DNA sequences at temperatures between 37-42° C. with recombinase enzymes and DNA binding proteins, but has not to date been validated in field settings.

Loop-mediated isothermal amplification (LAMP) can be used in a similar manner to RPA and can potentially detect infectious agents from multiple biological sources. Similar to PCR, LAMP can detect sections of DNA found in infectious pathogens. LAMP overcomes the restrictions placed on other diagnostic methods by providing the following four advantages: i) amplifies DNA under a single temperature incubation of ˜65° C., ii) assay specificity due to the use of four to six DNA primers (which can be designed using freely available software PrimerExplorer), iii) achieves limit of detections similar to conventional PCR, and iv) requires only a simple heating element for assay operation as opposed to complex thermocyclers.

While LAMP does show promise as an effective field diagnostic tool, a major limitation of using LAMP as a mainstream assay for pathogen screening is the occurrence of false positives—either due to poor reagent handling or carryover contamination from previous experiments. Additionally, the accuracy of LAMP is heavily dependent on the primers used and, prior to this disclosure, optimal primer sets had yet to be identified. Indeed, designing LAMP primers has proven challenging.

There remains a need to provide a cost-effective, rapid, and accurate diagnostic assay to detect BRD. While some conventional assays potentially show promise as tools in the diagnosis of BRD, portable molecular diagnostic assays for detecting the presence of the primary bacterial pathogens for BRD in bovine samples are needed that provide field testing capabilities.

### BRIEF SUMMARY

Loop-mediated isothermal amplification (LAMP) assays are provided. In at least one embodiment, such assays can comprise at least one LAMP primer set that targets a deoxyribonucleic acid (DNA) fragment of a pathogen associated with a bovine respiratory disease (BRD) in a sample (e.g., a bovine nasal sample or a bovine water sample taken from a trough from which the cattle drink, for example), wherein the assay allows for single-step identification of the pathogen. The pathogen associated with BRD can be, for example, a bacterium (e.g., bacterium or a bacterial mycoplasma), fungus, virus, or any other infectious agent associated with BRD. In certain embodiments, the pathogen associated with BRD is selected from the group consisting of Pasteurella multocida (P. multocida), Mannheimia haemolytica (M. haemolytica), and Histophilus somni (H. somni). In certain embodiments, the at least one LAMP primer set is one or more of primer sets A-AAAA in Tables 2 and 3.

In certain embodiments, the pathogen is P. multocida and the targeted DNA fragment comprises a gene selected from the group consisting of kmt1, ompP1, and omp16. In certain embodiments, the pathogen is M. haemolytica and the targeted DNA fragment comprises a gene selected from the group consisting of rsmL, rsmC, and lktA. In certain embodiments, the pathogen is H. somni and the targeted DNA fragment comprises a gene selected from the group consisting of lolA, lolB, and lppB. Each assay can also include various primer sets drawn to any combination of the foregoing. In certain embodiments, each LAMP primer set is at least about 98% specific to the targeted DNA fragment.

In an exemplary embodiment, the at least one LAMP primer set comprises a primer set that targets a kmt1 fragment of P. multocida, the at least one LAMP primer set comprises a primer set that targets a lolB fragment of H. somni, and/or the at least one LAMP primer set comprises a primer set that targets a rsmL fragment of M haemolytica.

Embodiments of the assay can process and provide a visual result in 60 minutes or less. For example, the visual result can be indicative of the presence or absence of the pathogen for BRD in the sample (e.g., being tested). In certain embodiments, each of the LAMP primer sets has a limit of detection (LoD) of at least 10³ copies/reaction.

The visual result can, for example, identify the type of pathogen present in the sample. In certain embodiments, the visual result is a color-coded or colorimetric result.

The targeted DNA fragment can comprise a gene associated with antibiotic resistance.

In certain embodiments, the assay comprises one or more indicators. Such indicators, for example, can comprise a pH- or magnesium-sensitive indicator. In some embodiments, the indicator comprises a magnesium-based indicator. In some embodiments, the indicator is a fluorescent indicator.

In certain embodiments, each LAMP primer set is coupled with a colorimetric reagent. The colorimetric reagent can be pH sensitive or magnesium sensitive, for example. In certain embodiments, the colorimetric reagent is phenol red.

In at least one embodiment, each LAMP primer set comprises one or more primers of SEQ ID NOS. 1-6, 25-30, 55-62, 99-110, and 147-158 or the primer sets embodied thereby.

In certain embodiments, each LAMP primer set comprises 4 to 6 primers.

Methods are also provided for identifying a pathogen associated with BRD in a sample and, in certain embodiments, the treatment thereof. In at least one embodiment, the method comprises: providing at least one LAMP primer set that targets a deoxyribonucleic acid (DNA) fragment of a targeted pathogen associated with a bovine respiratory disease (BRD) in a sample; obtaining a sample from a subject; combining the sample and the at least one LAMP primer set into a mixture; heating the combination to initiate amplification of the targeted DNA fragment; and detecting a visual result in the heated combination indicative of the presence or absence of the targeted pathogen for BRD in the sample. There, the at least one LAMP primer set can be, for example, one or more of primer sets A-AAAA in Tables 2 and 3.

In some cases, the methods can provide a visual result in 60 minutes or less of initiating the heating step and the sample is a bovine nasal sample (or a bovine water sample).

The step of detecting a visual result can also further comprise measuring a relative clarity of the heated combination using a turbidimeter; and analyzing colorimetric data in the visual result using one or more of a fluorescent reader, an ultraviolet light reader, or camera. The visual result can be indicative of the presence or absence of the targeted pathogen. In certain embodiments, if the visual result is indicative of the presence of a targeted pathogen, the method further comprises treating the subject (or cohort (e.g., herd)) for the targeted pathogen.

In certain embodiments of the method, the at least one LAMP primer set is coupled with a colorimetric reagent that is pH sensitive or magnesium sensitive. For example, the colorimetric agent can be phenol red.

In certain embodiments of the method, the at least one LAMP primer set targets a kmt1 fragment of P. multocida or a lolB fragment of H. somni. Further, the at least one LAMP primer set can comprise a first primer set targeting a kmt1 fragment of P. multocida and a second primer set targeting a lolB fragment of H. somni, or any combination of the primer sets identified herein.

Certain embodiments hereof comprise kits (e.g., portable kits). A nonlimiting example of such a kit can comprise at least one LAMP primer set that targets a DNA fragment of a pathogen associated with a BRD in a sample, at least one swab for obtaining the sample, and a heating element to initiate amplification of the targeted DNA fragment when the at least one LAMP primer set and the sample are combined.

In at least one embodiment, the kit can further comprise a fluorescent indicator, and a fluorescent reader, an ultraviolet light reader, or a camera to provide color metric result data indicative of the presence or absence of a targeted pathogen in the sample. In at least one embodiment, the at least one primer set is coupled with a colorimetric reagent that is pH sensitive or magnesium sensitive. In certain embodiments, the colorimetric agent is phenol red.

In certain embodiments, the at least one swab comprises a nasal swab and the kit further comprises a sealable container with a transport media therein. In certain embodiments, the heating element is a water bath.

The kits hereof can be portable and capable of use in a non-laboratory setting (e.g., the field).

The LAMP primer set(s) of the kit can be any of the LAMP primer sets described herein (or combination thereof). For example, the at least one LAMP primer set can target a kmt1 fragment of P. multocida or a lolB fragment of H. somni. Additionally or alternatively, the at least one LAMP primer set can comprise a first primer set targeting a kmt1 fragment of P. multocida and a second primer set targeting a lolB fragment of H. somni.

### BRIEF DESCRIPTION OF THE DRAWINGS

The disclosed embodiments and other features, advantages, and disclosures contained herein, and the matter of attaining them, will become apparent and the present disclosure will be better understood by reference to the following description of various exemplary embodiments of the present disclosure taken in conjunction with the accompanying drawings, figures, and tables, wherein:

FIG. 1 displays limit of detection (LoD) characterization data of assay primer sets of the present disclosure, wherein 5 μL of gDNA (1×10⁰ to 1×10⁵ copies/reactions) were added to reactions (20 μL reagents) in triplicate and incubated for 60 minutes at 65° C., and fluorescent intensities of primer set replicates were extracted at 45 minutes and compared to a threshold fluorescent intensity (28%) determined from ROC analysis; with highlighted cells representing reactions crossing the threshold considered positive (the lowest concentration at which all three replicates amplify is the LoD, noting lktAlktA.3 seemed to form dimers in water which led to false amplification (this is inhibited in liquid amies media);

FIG. 2 is a heat map of selected primer sets tested against 6 different isolates of bovine respiratory disease complex (BRD)-associated pathogens (cross-isolate data); isolates of P. multocida, M haemolytica, and H. somni were used (the initials on the x-axis refer to the bacteria genus and species, the numbers refer to a different strain as labelled by Indiana Animal Disease Diagnostic Lab), LAMP reactions were run in real-time (RT) in with a RT thermal cycler at 65° C., and fluorescence intensities were selected at 30 minutes (longest reaction time of 9 selected primer sets) to be plotted on the heat map; three replicates of each reaction were run and displayed individually on the map with water used as a negative control;

FIG. 3 is a receiver operator characteristic (ROC) curve illustrating true positive rate (TPR) and false positive rate (FPR) of BRD a loop-mediated isothermal amplification (LAMP) assay with the cross-isolate data as presented in FIG. 2;

FIG. 4 shows graphical amplification data of P. multocida genomic DNA (gDNA) present in water and DNA-spiked liquid amies; water and liquid amies samples were spiked with various concentrations of water-suspended DNA extracts (P. multocida, M haemolytica, and H. somni) to generate serial dilutions (1.0×10⁰ to 1.0×10⁵ copies of DNA/reaction), and ran through quantitative LAMP (qLAMP) assays with P. multocida specific primer sets for 60 minutes at 65° C. with water and water-spiked liquid amies used as negative controls;

FIG. 5 shows graphical amplification data of M. haemolytica gDNA present in water and DNA-spiked liquid amies; water and liquid amies samples were spiked with various concentrations of water-suspended DNA extracts (P. multocida, M haemolytica, and H. somni) to generate serial dilutions (1.0×10⁰ to 1.0×10⁵ copies of DNA/reaction), and ran through qLAMP assays with M haemolytica specific primer sets for 60 minutes at 65° C., with water and water-spiked liquid amies used as negative controls;

FIG. 6 shows graphical amplification data of H. somni gDNA present in water and DNA-spiked liquid amies; water and liquid amies samples were spiked with various concentrations of water-suspended DNA extracts (P. multocida, M haemolytica, and H. somni) to generate serial dilutions (1.0×10⁰ to 1.0×10 5 copies of DNA/reaction) and ran through qLAMP assays with H. somni specific primer sets for 60 minutes at 65° C., with water and water-spiked liquid amies used as negative controls;

FIG. 7 shows graphical qLAMP amplification curves for LAMP primer sets that target P. multocida, where 5 μL of gDNA from P. multocida, M haemolytica, and H. somni (0.2 ng/uL) was added to separate reactions in quadruplicate and incubated for 60 minutes at 65° C. and nuclease-free water was used as a negative control (Blue lines (labeled P): P. multocida gDNA reactions; Orange lines (labeled M): M. haemolytica gDNA reactions; Dark Green lines (labeled H): H. somni gDNA reactions; Black lines (labeled W): Water reactions);

FIG. 8 shows graphical qLAMP amplification curves for LAMP primer sets that target M haemolytica, where 5 μL of gDNA from P. multocida, M haemolytica, and H. somni (0.2 ng/uL) was added to separate reactions in quadruplicate and incubated for 60 minutes at 65° C. and nuclease-free water was used as a negative control (Blue lines (labeled P): P. multocida gDNA reactions; Orange lines (labled M): M. haemolytica gDNA reactions; Dark Green lines (labeled H): H. somni gDNA reactions; Black lines (labeled W): Water reactions);

FIG. 9 shows graphical qLAMP amplification curves for LAMP primer sets that target H. somni, where 5 μL of gDNA from P. multocida, M haemolytica, and H. somni (0.2 ng/uL) was added to separate reactions in quadruplicate and incubated for 60 minutes at 65° C. and nuclease-free water was used as a negative control (Blue lines (labeled P): P. multocida gDNA reactions; Orange lines (labeled M): M. haemolytica gDNA reactions; Dark Green lines (labeled H): H. somni gDNA reactions; Black lines (labeled W): Water reactions);

FIG. 10A is an image collected using the Epson Perfection V800 Photo scanner and background whitened using the ImageJ brightness/contrast setting that illustrates how a change in pH affects the colorimetric gradient resulting from the colorimetric reagents described herein;

FIG. 10B is a graphical representation of the average colorimetric absorbance ratios associated with FIG. 10A from three cycles collected with the CLARIOstar Plus;

FIGS. 11A-11C are graphical representations of the workflows described, with FIG. 11A illustrating a workflow where three different primers were screened through the limit of detection (LOD) study, with the best-selected primers in each species highlighted inside the black rectangle; FIG. 11B illustrating a workflow where several combinations of DNA were diluted in water and tested in a lab environment to study off-target behavior in pH-sensitive colorimetric reactions; and FIG. 11C illustrating a LAMP assay conducted on-farm with a prepared colorimetric master-mix and later repeated in-lab (a precision cooker was used as a heating device to confirm the ability of the assay in a resource-limited setting), with PM=Pasteurella multocida, MEI=Mannheimia haemolytica, and HS=Histophilus somni;

FIG. 12 is an LOD table showing the selection process for primers for each gene target, with the recorded numbers indicating the colorimetric absorbance ratio of absorbance measured at 430 nm to 520 nm and the highlighted cells corresponding to colorimetric absorbance ratios higher than 3.0 (missing data indicates conditions that were not tested);

FIG. 13 shows LAMP colorimetric results with PM, MEI, and HS gDNA present at 60 minutes (water-suspended DNA extracts of the corresponding gDNA were added to water to generate two-fold serial dilutions (10,000 to 78.125 copies of DNA/reaction)), with the results indicating that at least ktm1 detects PM, rsmL detects MH, and lolB detects HS (in the black and white version shown herein, the darker plates represent a pink or dark orange color and the lighter plates represent a yellow color). The DNA were added to qLAMP assays with the primer sets being tested for 60 minutes at 65° C., with water used as a negative control. Images were collected using the Epson Perfection V800 Photo scanner and the background was whitened using the ImageJ brightness/contrast setting;

FIG. 14 show quantitative results of PM, MH, and HS gDNA present in water as described in FIG. 13 (for primers lolA, lolB, and lppB, the data points at minute 59 were excluded due to them being negative values potentially due to instrument error; each panel had three replicates);

FIG. 15 shows a LOD analysis python script, ColorimetriAnalysis.py, which was used to analyze primer set screening data and is executable by importing the file and executing “getPrimerSummer(filename,output)” and providing the file name or path along with the output.xls file name or path; the resulting Excel file will contain two sheets—one containing the final primer scoring and the other containing intermediate calculations for each concentration in each primer set;

FIG. 16 is an image of representative colorimetric results for positive and negative qLAMP reactions, with positives taken from qLAMP reactions run with 10,000 copies of DNA per reaction and negatives taken from qLAMP reactions without DNA, kmt1 primers used to detect PM, rsmL primers used to detect MH and lolB primers used to detect HS, all samples imaged at 60 minutes using an Epson Perfection V800 Photo scanner and the background whitened using the ImageJ brightness/contrast setting;

Figure is an 17 image of LAMP colorimetric results with different combinations of PM, MEI, and HS gDNA present in water at 60 minutes, where water-suspended DNA extracts at 1,250 copies of DNA per reaction were added to qLAMP assays with the primer sets being tested for 60 minutes at 65° C., kmt1 detecting PM, rsmL detecting MH, and lolB detecting HS; DNA-free water used as a negative control; and images collected using the Epson Perfection V800 Photo scanner and background whitened using the ImageJ brightness/contrast setting;

FIG. 18 shows quantitative results of LAMP detection of the different combinations of PM, MH, and HS gDNA present in water at 60 minutes of FIG. 17, with absorbance ratios above 3.0 considered positive and absorbance ratios below 3.0 considered negative; DNA-free water used as negative control; and where each panel had nine replicates;

FIG. 19 shows ROC curves for each selected primer set, with the graphs illustrating the TPR and FPR of the qLAMP assay for each primer using the combinations of BRD bacteria presented in FIGS. 17 and 18 (TPR calculated as TP/(TP+FN) and FPR calculated as FP/(FP+TN) (false positive=FP; true negative=TN; true positive=TP; false negative=FN);

FIG. 20 illustrates a LAMP procedure performed on a farm, with FIG. 20A showing a nasal sample being extracted from a steer; FIG. 20B showing the extracted mucus on the swab being diluted to 200 μL of water; FIG. 20C showing 5 μL of resuspended nasal swab solution used as a sample; FIG. 20D showing 5 μL of resuspended nasal swab solution being added to pre-prepared colorimetric LAMP tubes with different primer sets; and FIG. 20E showing the tubes being incubated for 60 minutes at 65° C. inside a precision cooker (previous experiments ensured the submersion of PCR tubes would not cause inward leaking);

FIG. 21 shows a top-down thermal image of a precision cooker used to heat the LAMP reactions for the LAMP water bath experiments described in the Examples, with a first cursor indicating the point of highest temperature (65.3° C.) which corresponds to the color white and a second cursor indicating the point of lowest temperature (23.6° C.) which corresponds to the color black and is outside the boundaries of the pressure cooker (central point is white and 65.2° C.; LAMP reactions were submerged in the water on the right side of the precision cooker where the temperature was most consistent);

FIG. 22A showing a 3D model of a PCR tubes holder used in the present Examples with two hanging parts for convenient placement of the tubes and three sets of eight tubes each; FIG. 22B showing a slider to cover the tubes from floating in the precision cooker; FIG. 22C showing a PCR tube holder.stl file; and FIG. 22D showing a slider to cover the tubes.stl file (units are millimeters);

FIG. 23 shows an image of colorimetric results from the on-farm LAMP detection of bacteria in unprocessed mucus collected from steers (see Example 15), with LAMP reactions run in polymerase chain reaction (PCR) tubes submerged in water at 65° C. inside a precision cooker, kmt1 detecting PM, rsmL detecting MH, and lolB detecting HS, the reactions run for 60 minutes, DNA-free water used as a negative control and reactions diluted in water (images collected using a Samsung Galaxy A50 and adjusted using the brightness/contrast tool on Image J and the white balance tool on Adobe Lightroom);

FIG. 24 shows an image of colorimetric results from the in-lab LAMP detection of bacteria in unprocessed mucus collected from steers (see Example 15), with LAMP reactions run in PCR tubes submerged in water at 65° C. inside a precision cooker, kmt1 detecting PM, rsmL detecting MH, and lolB detecting HS, the reactions run for 60 minutes, DNA-free water used as a negative control and reactions diluted in water (images collected using an Epson Perfection V800 Photo scanner and adjusted using the brightness/contrast tool on Image J and the white balance tool on Adobe Lightroom);

FIG. 25 shows PCR confirming results run on 1% agarose gel with 1 kbp DNA ladder as a marker for 5 steers with 3 different primers corresponding to PM, MH, and HS, with PCR conducted with the extracted genomic DNA from the mucus obtained from respective steers, PCR performed using Thermo Fisher Phusion™ Hihg-Fidelity DNA Polymerase (F-530XL), the extracted genomic DNA used as a template for the PCR reaction (expected gene sizes ere PM: ompP1—1180 bp, MH: iktA—1932 bp, and HS: IppB—404 bp); and

FIG. 26 shows a table displaying on-farm versus in-lab Hue values from the experiment of Example 15 (precision cooker experiment) and as shown on-farm in FIG. 23 and in-lab as shown in FIG. 24; for both LAMP experiments and PCR, the highlighted cells indicate positive samples and the white cells indicate negative samples (between farm LAMP and PCR, 2 out of 3 LAMP reactions with the same result as PCR were considered agreement).

As such, an overview of the features, functions and/or configurations of the components depicted in the various figures will now be presented. It should be appreciated that not all of the features of the components of the figures are necessarily described and some of these non-discussed features (as well as discussed features) are inherent from the figures themselves. Other non-discussed features may be inherent in component geometry and/or configuration. Furthermore, wherever feasible and convenient, like reference numerals are used in the figures and the description to refer to the same or like parts or steps. The figures are in a simplified form and not to precise scale.

### DETAILED DESCRIPTION

For the purposes of promoting an understanding of the principles of the present disclosure, reference will now be made to the embodiments illustrated in the drawings, tables, and figures and specific language will be used to describe the same. It will nevertheless be understood that no limitation of the scope of this disclosure is thereby intended.

The present disclosure includes various assays, kits, and methods to target and/or detect and/or treat the presence or absence of bovine respiratory disease complex (BRD)-associated pathogens, such as to diagnose and treat BRD. These assays (and methods of treatment using such assays) can be portable, disposable, and capable of providing fast and accurate results in the field without the need for a laboratory and other complex equipment. The terms “treat,” “treating,” “treated,” or “treatment” (with respect to a disease or condition) is an approach for obtaining beneficial or desired results including and preferably clinical results and includes, but is not limited to, one or more of the following: improving a condition associated with a disease, curing a disease, lessening severity of a disease, delaying progression of a disease, alleviating one or more symptoms associated with a disease, increasing the quality of life of one suffering from a disease, prolonging survival and/or prophylactic or preventative treatment.

Additionally the assays presented herein provide rapid and accurate results (as compared to conventionally available assays and other methodologies). Perhaps more specifically, the novel primer sets of the assays, kits, and methods hereof increase testing accuracy (e.g., at or about 99% analyticalsensitivity and at or about 89% analytical specificity) and decrease testing time to less than 45 minutes, thus providing fast and accurate results.

In at least one embodiment, a portable assay or method using the same comprises a loop-mediated isothermal amplification (LAMP) assay that utilizes novel primers (e.g. primer sets). Also disclosed herein are detection methods using LAMP assays that can specifically target and detect the presence of BRD-associated bacterial pathogens such as Pasteurella multocida, Mannheimia haemolytica, Histophilus somni, and Mycoplasma bovis from bovine nasal or nasopharyngeal samples to provide a BRD diagnosis. Accordingly, the assays, kits and methods hereof can be used to rapidly and accurately diagnose BRD in the field such that treatment, where desired, can be administered.

LAMP uses 4-6 primers that can recognize 6-8 distinct regions of target deoxyribonucleic acid (DNA) for a highly specific amplification reaction. A strand-displacing DNA polymerase initiates synthesis and two specifically designed primers form “loop” structures to facilitate subsequent rounds of amplification through extension on the loops and additional annealing of primers. DNA products are typically long (>20 kb) and formed from numerous repeats of the short (80-250 bp) target sequence, connected with single-stranded loop regions in long concatamers. These products are not typically appropriate for downstream manipulation, but the achievable target amplification can be so extensive that numerous modes of detection are possible.

Real-time fluorescence detection using intercalators or probes, lateral flow, and agarose gel detection, for example, are all directly compatible with LAMP reactions. Instrumentation for LAMP typically requires consistent heating to the desired reaction temperature and, where desired, real-time fluorescence for quantitative measurements. Optimized settings for running LAMP assays on isothermal instruments are known in the art, and the assay can be performed using the techniques described in detail in at least Notomi, T. et al., “Loop-mediated isothermal amplification of DNA,” Nucleic Acids Res. 2000, Jun. 15; 28(12): e63 (doi: 10.1093/nar/28.12.e63) and Nagamine, K et al., “Accelerated reaction by loop-mediated isothermal amplification using loop primers,” Mol. Cell. Probes 2002; 16: 223-229, both of which are incorporated herein by reference in their entireties.

In certain instances, LAMP can be so prolific that the products and byproducts of these reactions can be visualized by the naked eye. For example, magnesium pyrophosphate produced during the reaction can be observed as a white precipitate or added indicators (e.g., calcein or hydroxynaphthol blue) can be used to signal a positive reaction or an indicative pH change.

In certain embodiments, the LAMP assay can be coupled with a colorimetric reagent that is sensitive to magnesium or pH and allows for visualization of the result with the naked eye and/or quantification using a camera. Such colorimetric reagents, for example, can include a phenol red. In certain embodiments, the LAMP assays hereof coupled with a colorimetric reagent has a limit of detection of 1250 copies of DNA per reaction, with an analytical specificity of 100%, and/or an analytical sensitivity in the range of 66.7% -100%. In an exemplary embodiment, the primers described herein are coupled with a composition comprising phenol red, such as, for example and without limitation, Warmstart® LAMP 2× Master Mix.

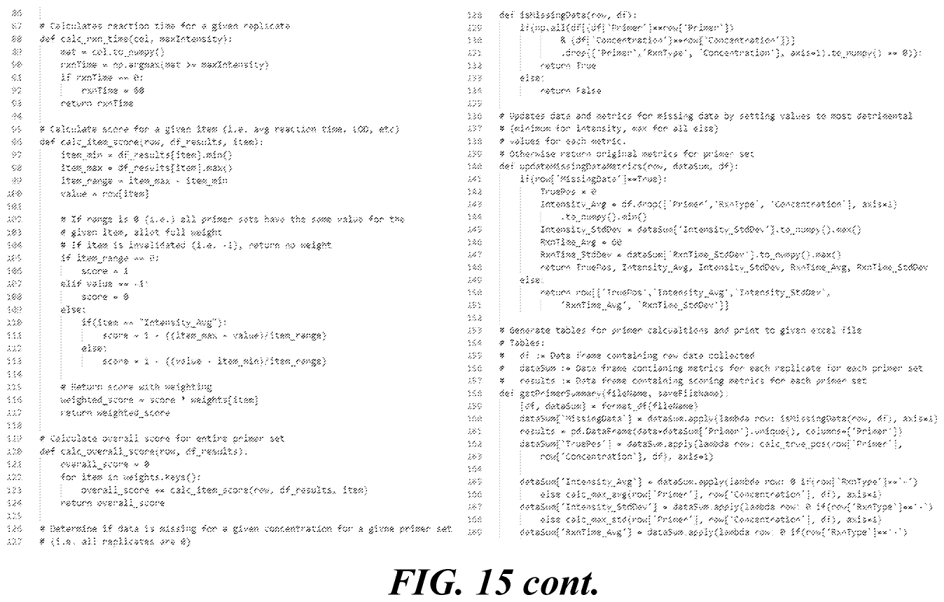

Warmstart® LAMP 2× Master Mix, which contains phenol red, is characterized by its transition from pink to yello as the LAMP reaction occurs and the pH decreases. Positive and negative results using the primers hereof for detection of BRD pathogens and the pink and yellow color distinction can be observed in FIG. 16.

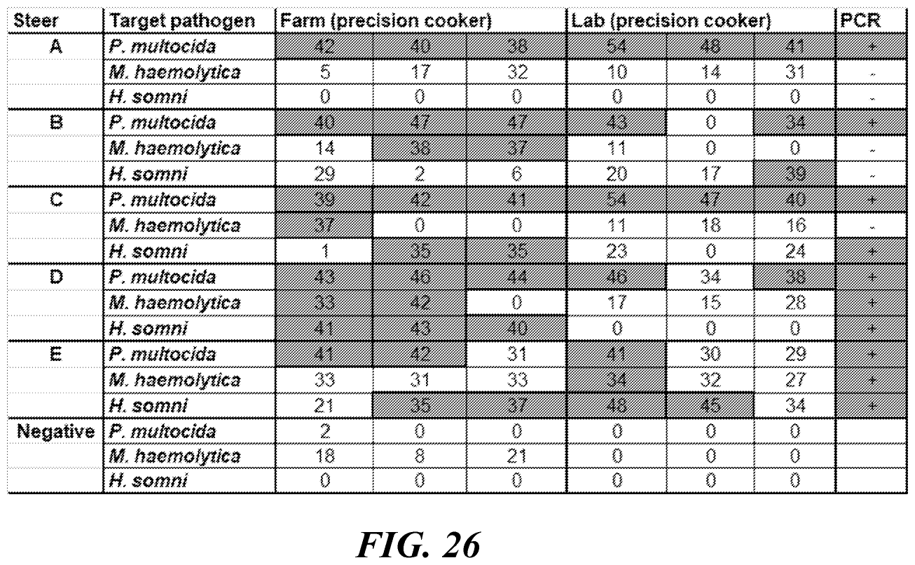

As noted above, while some work has been done to determine the presence of BRD pathogens, conventional precedent does not allow for BRD pathogens to be accurately detected in a field setting. The colorimetric LAMP assays for BRD pathogens provided herein however, are accurate and sensitive. Indeed, Table 7 (below) and FIG. 26 highlight the concordance between conducting the assays on-farm and in a laboratory setting (in-lab). Surprisingly, as described in Example 15 below, there was a higher concordance between the on-farm colorimetric LAMP and in-lab Polymerase chain reaction (PCR), as compared to on-farm and in-lab colorimetric LAMP, which suggests that the mucus transportation from the farm to the lab may lead to DNA degradation that results in false negatives in LAMP reactions (but not in PCR).

The colorimetric LAMP assays hereof offer at least six advantages: (1) they can be conducted on the farm/in the field using a simple consumer-grade water bath; (2) they can provide a visual readout and, thus, allow for analysis with the naked eye; (3) they provide a response within 60 minutes; (4) they do not require sample processing (e.g., extraction of nucleic acids); (5) they can detect at least the pathogens P. multocida and H. somni with a high degree of accuracy (100% and 96%, respectively); and (6) they utilize a simple non-invasive nasal swab for sampling.

LAMP is well-suited for point-of-care and field diagnostics using all manner of sample types. Further, the LAMP reaction is robust and tolerant of inhibitors, allowing for crude sample prep and minimal nucleic acid purification, if desired.

In at least one illustrative LAMP assay hereof, the assay is for single-step identification of a targeted infectious agent in a sample. The LAMP assay comprises at least one novel LAMP primer set that targets a DNA of a pathogen associated with BRD in a sample. The assay not only allows for detection of the targeted pathogen within a sample, but also the distinction thereof from other pathogens in a single step. Further, the LAMP assays hereof can achieve at least a sensitivity and specificity of about 97.2% and 90.9%, respectively, which is a significant increase of sensitivity as compared to existing LAMP models. In certain embodiments, the novel LAMP primer sets are also designed to enhance amplification speed of the assay such that the LAMP assays hereof are more rapid than conventional LAMP assays.

In at least one embodiment, the LAMP primer set comprises F3, B3, FIP, BIP primers. Additionally, the LAMP primer set can also comprise loop primers (e.g., labelled as LF (loop forward) and/or LB (loop backward)).

The pathogen associated with BRD to be detected by the LAMP assays hereof can be a bacterium (including, without limitation, a bacterial mycoplasma), a fungus, a virus, and/or any other infectious agent that is associated with a BRD. In at least one embodiment, treatment and detection methods using a LAMP assay and primers, may specifically detect the presence of BRD-causing bacteria (or bacterial mycoplasma) such as P. multocida, M haemolytica, and H. somni, such as in 60 minutes or less (e.g., less than 60 minutes, less than 55 minutes, less than 50 minutes, or less than 45 minutes).

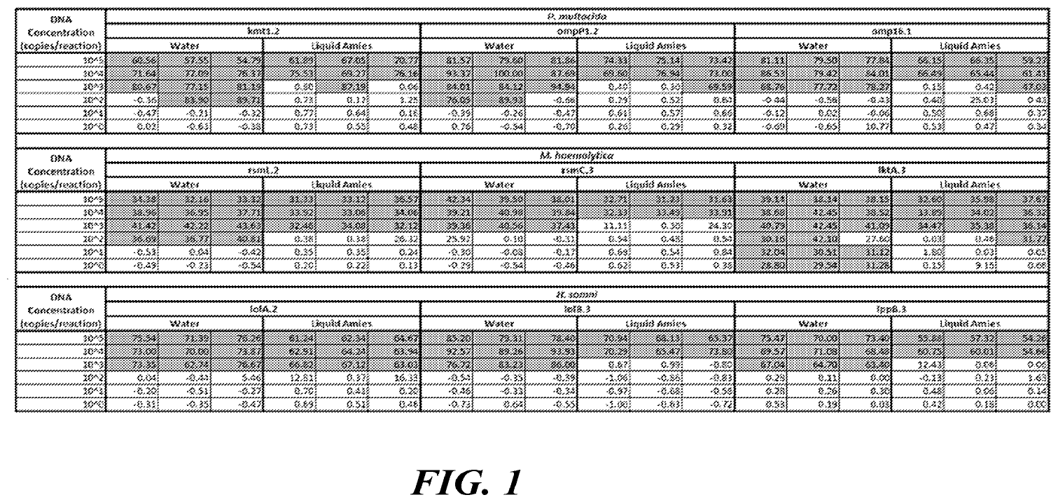

The targeted DNA of each pathogen is, preferably, a DNA segment or region that has little to no homology with non-targeted BRD-associated pathogens. While some such gene targets are known, others such as those listed in FIG. 1 and Table 1 below were newly identified by the present investigators.

TABLE 1
List of gene targets for BRD bacterial pathogens

| Bacteria Name | Candidate Gene Name | Sequence/Genbank ID |
|---|---|---|
| Pasteurella multocida | kmt1 | AF016259.1 |
| | ompP1 | QGV32322.1 |
| | omp16 | AJ271673 |
| Mannheimia haemolytica | rsmL | QEC27547.1 |
| | rsmC | QEC27614.1 |
| | lktA | QEC25656.1 |
| Histophilus somni | lolA | ACA31013.1 |
| | lolB | ACA31225.1 |
| | lppB | ACA32113.1 |

In certain embodiments, the targeted gene of the pathogen can comprise a gene associated with antibiotic resistance. Targeting such genes using the assays hereof can allow for timely identification and diagnosis of drug-resistant strains of pathogens present within a herd or other cattle population. Accordingly, any such infected cattle can be removed from the population and/or treated before an outbreak occurs, which can improve overall anti-infective management.

Each LAMP primer set of the assay is designed to target and amplify the targeted gDNA from a targeted pathogen, while maintaining little to no amplification of other pathogens or negative samples. Each LAMP primer set can include 4 to 6 DNA primers (however the number of primers used can be modified, as desired). In some embodiments (for example, where the pathogen(s) to be detected are a bacterium), the LAMP primer set can target an oppD/F genomic region of the pathogen associated with BRD. This domain has been reported to be capable of discriminating M bovis, for example, from the highlight homologous (at the genome level) species Mycoplasma agalactiae.

In other embodiments, the LAMP primer set(s) hereof each comprise a LAMP primer set listed in Tables 2 or 3. In certain embodiments, the LAMP primer set(s) comprise one or more primers of any of SEQ ID NOS. 1-6, 25-30, 55-62, 99-110, and 147-158.

TABLE 2
Primer sets for targeting BRD-associated pathogens.

| Primer | SEQ | | |
|---|---|---|---|
| Set | ID | Primer | |
| ID: | NO: | Set | Sequence (5′ to 3′) |
| A | 1 | kmt1.2 F3 | GAATCAAGCGGTCACAG |
| | 2 | kmt1.2 B3 | CACTCACAACGAGCCATA |
| B | 3 | kmt1.2 FIP | AGAGCAGTAATGTCAGCACAATATTAAAGACAGCAATTTCGA |
| | | | GCA |
| | 4 | kmt1.2 BIP | CGCTATTTACCCAGTGGGGCGCCATTTCCCATTTCAAGTG |
| C | 5 | kmt1.2 LF | CGTAAAGCCCCACCATTGTT |
| | 6 | kmt1.2 LB | ACCGATTGCCGCGAAATTGAGT |
| D | 7 | ompP1.2 | GCAATTTATGTGGACCCAAAT |
| | | F3 | |
| | 8 | ompP1.2 | AATCGGTTTTACCGCCTA |
| | | B3 | |
| E | 9 | ompP1.2 | TCGGAACTAACGCATTCGGCTTAACTTCACCAATGCCAGG |
| | | FIP | |
| | 10 | ompP1.2 | ATCCAATTAACGAAAAATTCGCTGTGCATATTTGTCATCAAAC |
| | | BIP | TCGG |
| F | 11 | ompP1.2 | CAATATTTTTATAGGCGAA |
| | | LF | |
| | 12 | ompP1.2 | GGCGGTGGATTGAATGTCAAC |
| | | LB | |
| G | 13 | omp16.1 | GGCGGTTATTCAGTACAAGA |
| | | F3 | |
| | 14 | omp16.1 | CATCTGCACGACGTTGAC |
| | | B3 | |
| H | 15 | omp16.1 | CGCATGTGCATCTAAAATTTGTACAGTTATAATACCGTGTATT |
| | | FIP | TCGGC |
| | 16 | omp16.1 | AATGCAACACCTGCAACGAACTAATGCGATGTTATATTCTGGT |
| | | BIP | |
| I | 17 | omp16.1 | CGATATTGTATTTATCGA |
| | | LF | |
| | 18 | omp16.1 | CGTTGTTGAAGGTAACACCGA |
| | | LB | |
| J | 19 | lktA.3 F3 | GTAACGACGGCAATGACC |
| | 20 | lktA.3 B3 | ATCTTTTAAGTTCGAATCAGAGA |
| K | 21 | lktA.3 FIP | TTGCCTTTACCGCCATCGATAAAGTAAAGGCGATGATATTCTC |
| | | | G |
| | 22 | lktA.3 BIP | GGTGGCAAGGGCGATGATATCATTGCCGTCAGAATCGG |
| L | 23 | lktA.3 LF | TCATCACCATTTCCACCA |
| | 24 | lktA.3 LB | TCGTTCACCGTAAAGGCGAT |
| M | 25 | rsmL.2 F3 | CGAAGACACTCGCCACAG |
| | 26 | rsmL.2 B3 | AACTTTTACCCCGGCTTGG |
| N | 27 | rsmL.2 FIP | AACGACCGCTTTCTGCTGTTCATTATTGCTGAGCCACTACGG |
| | 28 | rsmL.2 BIP | TGCGTTAATTTCCGATGCCGGACGGCAATGACGGACAAGA |
| O | 29 | rsmL.2 LF | GTGCAAGGCGAAAAACGGTTTTTTA |
| | 30 | rsmL.2 LB | GCCACTGATTAGCGACCCG |
| P | 31 | rsmC.3 F3 | CGGCAGACGTACTTTGGC |
| | 32 | rsmC.3 B3 | ATGCGTTTGCGACAATTCG |
| Q | 33 | rsmC.3 FIP | GTGGAATGGTGGGTTGGAGACA-AGAGGGGGAAGTGGTAGC |
| | 34 | rsmC.3 BIP | ACGGGGTCGATACCGCCTAC-GCTCACCGCCTTTGGTTA |
| R | 35 | rsmC.3 LF | CCGTTCATTAATGTGAGAGAACACA |
| | 36 | rsmC.3 LB | TGGAGGAGTTGATTTTCCAAGCT |
| S | 37 | lppB.3 F3 | AGCACAAAAAATACTGAGCA |
| | 38 | lppB.3 B3 | AGAGAAGGAGATTATTTGGAATG |
| T | 39 | lppB.3 FIP | TGTTGCCCATACTTCTAAGGTTAAATTTTGTAGCCTCAGTTTTC |
| | | | AAGC |
| | 40 | lppB.3 BIP | ACCGAATAAACAAAGCTATCCGATTTTTGCTGATTTTGCTAAT |
| | | | GCGG |
| U | 41 | lppB.3 LF | TTTCTCTGCTTCATAACC |
| | 42 | lppB.3 LB | CGCACTTTCTTTGATAACTCTCGT |
| V | 43 | lolA.2 F3 | AGTAATGTAACTTGGGCAAAT |
| | 44 | lolA.2 B3 | GCAATAATTTGACTTTCTTGAGG |
| W | 45 | lolA.2 FIP | CACTTGTTGTGTATAGTCAGCACTTCGGTTAATGAGTTACAAA |
| | | | ATCG |
| | 46 | lolA.2 BIP | ATGCACAGGGAAAAAAAATACAGCTGTTTCATTGTCCATACG |
| | | | AAAT |
| X | 47 | lolA.2 LF | ACACATCAATTTTATTTAA |
| | 48 | lolA.2 LB | GGAAAAATACAACTCAAACGT |
| Y | 49 | lolB.3 F3 | GCTACGTGAAATGATTGGTATC |
| | 50 | lolB.3 B3 | CTTTTCAGAAGAATATCTTTGGGTA |
| Z | 51 | lolB.3 FIP | GCCGACCTGATAATCTGAATTTTCATATTCCATTACAACAAAT |
| | | | AGGGAAC |
| | 52 | lolB.3 BIP | GCAAGCTTTACTTATTCAGTTGAGGATGCTTTGATCTGTTCGA |
| | | | TAG |
| AA | 53 | lolB.3 LF | CTGGTTGACCTTTTAGCC |
| | 54 | lolB.3 LB | GAAGTTTGGAGTGCTGAC |

TABLE 3
Additional primer sets for targeting respective
BRD-associated pathogens to be screened.

| | SEQ | | |
|---|---|---|---|
| Primer | ID | Primer | |
| Set ID | NO: | Set | Sequence (5′ to 3′) |
| BB | 55 | kmt1.1 LF | CGACCATCGGTTGCATTTC |
| | 56 | kmt1.1 LB | TGGCATTGCATGGCTATCA |
| CC | 57 | kmt1.3 F3 | CGCTGATTAATATTGTGCTGA |
| | 58 | kmt1.3 B3 | CCCAACAAAACTGTGCTT |
| DD | 59 | kmt1.3 FIP | TTCGCGGCAATCGGTTCATTCATTACTGCTCTATCCGCTAT |
| | 60 | kmt1.3 BIP | TATGCCACTTGAAATGGGAAATGGCCAAATAAAAGACTAC |
| | | | CGACAA |
| EE | 61 | kmt1.3 LF | ACCGCCCCACTGGGTAA |
| | 62 | kmt1.3 LB | TTTTATGGCTCGTTGTGAGTGG |
| FF | 63 | ompP1.1 F3 | TTCCAACTAGCAGAAGTGT |
| | 64 | ompP1.1 B3 | CCTGGCATTGGTGAAGTT |
| GG | 65 | ompP1.1 FIP | ACTGCCGCATTATCTGCAATCCAACATCCGGTTTAGGGC |
| | 66 | ompP1.1 BIP | GCCACGAACCCAGCATTAATACATAAATTGCCCCAACAG |
| HH | 67 | ompP1.1 LF | GGCTTCCCCCGCATAAGCA |
| | 68 | ompP1.1 LB | CTTATTGAAACAACCTGAA |
| II | 69 | ompP1.3 F3 | GCTCTTGCACCTGCATTA |
| | 70 | ompP1.3 B3 | GGTCTTACCGTCCGTACC |
| JJ | 71 | ompP1.3 FIP | CGGTAGCGGTAAATGTAAAGTACCAGTCCTCAAGGTATTAC |
| | | | TGC |
| | 72 | ompP1.3 BIP | TTCTGGCTATCATAAAATGACCGATAACTCTTTAAATTTGCT |
| | | | CCATTG |
| KK | 73 | ompP1.3 LF | GGGATTTCTTTGCCACCTGT |
| | 74 | ompP1.3 LB | TTTTGCAATGCACTATAGCT |
| LL | 75 | omp16.2 F3 | CAGTACAAGATTTACAACAACG |
| | 76 | omp16.2 B3 | AAATAATGTTTAACTGCATCTGC |
| MM | 77 | omp16.2 FIP | CGCATGTGCATCTAAAATTTGTACATTATAATACCGTGTATT |
| | | | TCGGC |
| | 78 | omp16.2 BIP | CGTTCTTAAATGCAACACCTGCGATGTTATATTCTGGTGTAC |
| | | | CG |
| NN | 79 | omp16.2 LF | CCTTCGATATTGTATTTATCG |
| | 80 | omp16.2 LB | TGTTGAAGGTAACACCGATGAAC |
| OO | 81 | omp16.3 F3 | GCGTTCTTAAATGCAACACC |
| | 82 | omp16.3 B3 | TATGCTAACACAGCACGA |
| PP | 83 | omp16.3 FIP | GACGTTGACCTAATGCGATGTTATGTTGTTGAAGGTAACAC |
| | | | CG |
| | 84 | omp16.3 BIP | CAAGCTGGTCAAGTATCAACAGTAGTAAGCTGCTTCATCGT |
| | | | G |
| QQ | 85 | omp16.3 LF | TTCTGGTGTACCGCGTTCA |
| | 86 | omp16.3 LB | CTTACGGTGAAGAGAAACCTGC |
| RR | 87 | lktA.1 F3 | TTCTTTAAAAACTGGGGCAA |
| | 88 | lktA.1 B3 | CAATAGCGGTTTGAATCGT |
| SS | 89 | lktA.1 FIP | CGCTTTGACTAAATCCTGTAAACCACTCTATATTCCCCAAA |
| | | | ATTACCAAT |
| | 90 | lktA.1 BIP | CCGAAGAGTTGGGGATTGAGGGCCTAAACTGGTTTGAGCT |
| TT | 91 | lktA.1 LF | TACCTTGTTCAGTATCA |
| | 92 | lktA.1 LB | AAGAACGCAATAATATTGCAA |
| UU | 93 | lktA.2 F3 | CCGAAGAGTTGGGGATTG |
| | 94 | lktA.2 B3 | GCATTTTGTACAATGCTTTCG |
| VV | 95 | lktA.2 FIP | CGGTTTGAATCGTGCCTAAACTGGTACAAAGAGAAGAACG |
| | | | CAAT |
| | 96 | lktA.2 BIP | CTATTGGCTTAACTGAGCGTGGTGGCCTGCTTTAGTTTTCTG |
| WW | 97 | lktA.2 LF | TTTGAGCTGTTGCAATAT |
| | 98 | lktA.2 LB | TTGTGTTATCCGCTCCACAAAT |
| XX | 99 | rsmL.1 F3 | ATTTAGTCGCCGCCGAAG |
| | 100 | rsmL.1 B3 | CCCGGGTCGCTAATCAGT |
| YY | 101 | rsmL.1 FIP | TGGTCGTGCAAGGCGAAAAACACAGCGGTTTATTGCTGAGC |
| | 102 | rsmL.1 BIP | ATGAACAGCAGAAAGCGGTCGTCGTTCCGGCATCGGAAAT |
| ZZ | 103 | rsmL.1 LF | GTTTTTTAATGCCGTAGT |
| | 104 | rsmL.1 LB | TTAGCAAAAGGGGAAAACATTGCGT |
| AAA | 105 | rsmL.3 F3 | TTTAGTCGCCGCCGAAGA |
| | 106 | rsmL.3 B3 | TGACGGACAAGATGAAAGCC |
| BBB | 107 | rsmL.3 FIP | ATGGTCGTGCAAGGCGAAAAACACAGCGGTTTATTGCTGA |
| | | | GC |
| | 108 | rsmL.3 BIP | TGAACAGCAGAAAGCGGTCGTTCGTTCCGGCATCGGAAAT |
| CCC | 109 | rsmL.3 LF | GTTTTTTAATGCCGTAGT |
| | 110 | rsmL.3 LB | ATTAGCAAAAGGGGAAAACATTGCG |
| DDD | 111 | rsmC.1 F3 | TGCGAGATGATTTTGCCCG |
| | 112 | rsmC.1 B3 | ACATTTCTTGCCCTGCTTGG |
| EEE | 113 | rsmC.1 FIP | CTCACTATGGTGGCGTGCGTATCGTGCGGAAAAGGTTGC |
| | 114 | rsmC.1 BIP | TTGAGTGCCGAGTTAGCGGTGACAGCCATTGTAGCAGTTGG |
| FFF | 115 | rsmC.1 LF | TCAAAATAGCTACTGAATAC |
| | 116 | rsmC.1 LB | AAGAACAAGCAGGAATGCC |
| GGG | 117 | rsmC.2 F3 | GGAATGCCAATTCCAACTGC |
| | 118 | rsmC.2 B3 | GCAAGCGGTAGGATTTCCAG |
| HHH | 119 | rsmC.2 FIP | CACCAGCTCGGTTTTCGCCGCAATGGCTGTCGCAATGC |
| | 120 | rsmC.2 BIP | CGAACCGTTCGGCAATATTGCC-TGTCGGCACGGTCTGTAG |
| III | 121 | rsmC.2 LF | TCAACATTTCTTGCCCTGCTT |
| | 122 | rsmC.2 LB | AATCGACTCTGCACGCCGTTG |
| JJJ | 123 | lppB.1 F3 | CACAAAAAATACTGAGCAGGTA |
| | 124 | lppB.1 B3 | AGAGAAGGAGATTATTTGGAATG |
| KKK | 125 | lppB.1 FIP | CTGTTGCCCATACTTCTAAGGTTA- |
| | | | GCCTCAGTTTTCAAGCGA |
| | 126 | lppB.1 BIP | TACCGAATAAACAAAGCTATCCGAT- |
| | | | GCTGATTTTGCTAATGCGG |
| LLL | 127 | lppB.1 LF | TTATGAAGCAGAGAAAAT |
| | 128 | lppB.1 LB | CGCACTTTCTTTGATAACTCTCGT |
| MMM | 129 | lppB.2 F3 | ACATTGGTACTGGAAAGCAA |
| | 130 | lppB.2 B3 | GCAGAGAAAATTTTAACCTTAGAAG |
| NNN | 131 | lppB.2 FIP | ATCAACGATACTCACATTGTTAGCTTTTTCTCACAGTAAAAT |
| | | | GTCAATTGC |
| | 132 | lppB.2 BIP | GTCACTGTTTTGCGTCTAAATAGTTTTCCTCGCTTGAAAACT |
| | | | GAGG |
| OOO | 133 | lppB.2 LF | GTAATTGACGACGTAG |
| | 134 | lppB.2 LB | AAAAAATACTGAGCAGGT |
| PPP | 135 | lolA.1 F3 | AAGTGACTGATGCACAGG |
| | 136 | lolA.1 B3 | CAAAACAAAGGGGGTATCG |
| QQQ | 137 | lolA.1 FIP | AGGTGTTTCATTGTCCATACGAAATGAAAAAAAATACAGCA |
| | | | AGGTAGT |
| | 138 | lolA. 1 BIP | TTGCAGATGGAAAAACATTATGGTTCTTAAGGTATCTTCAA |
| | | | CCCAATT |
| RRR | 139 | lolA.1 LF | GGACGTTTGAGTTGTATTT |
| | 140 | lolA.1 LB | CGATCCTTTTGTTGAGCAAG |
| SSS | 141 | lolA.3 F3 | TCAAACGTCCTAATTTATTTCGT |
| | 142 | lolA.3 B3 | CGTATCGGATTTTTGCTCAA |
| TTT | 143 | lolA.3 FIP | ACTTGCTCAACAAAAGGATCGTAGAATGGACAATGAAACA |
| | | | CCTCA |
| | 144 | lolA.3 BIP | AACAGCAAATTGGGTTGAAGATACCTGTTGCCAATGACTTG |
| | | | GAT |
| UUU | 145 | lolA.3 LF | TCCATCTGCAATAATTTGACT |
| | 146 | lolA.3 LB | GCGATACCCCCTTTGTTTTGC |
| VVV | 147 | lolB.1 F3 | CATCAGCTTTCAGCAGTTC |
| | 148 | lolB.1 B3 | AAATGATTTATGCCGACCTG |
| WWW | 149 | lolB.1 FIP | GTTGATATCTTTTGTTGAACGCTGATTTCACCTTTCGGTATT |
| | | | ACTGT |
| | 150 | lolB.1 BIP | AACTTGCTACGTGAAATGATTGGTTGAATTTTCATCTGGTTG |
| | | | ACCT |
| XXX | 151 | lolB.1 LF | TTCCTTTATGGTCTGA |
| | 152 | lolB.1 LB | CTCTATTCCATTACAACAAAT |
| YYY | 153 | lolB.2 F3 | AACGTTTTTCCACTCGCT |
| | 154 | lolB.2 B3 | CATTTCACGTAGCAAGTIGT |
| ZZZ | 155 | lolB.2 FIP | TGAACTGCTGAAAGCTGATGAAAACTTGGCAATATAACAAT |
| | | | CCG |
| | 156 | lolB.2 BIP | AGTTTCACCTTTCGGTATTACTGTTTGATATCTTTTGTTGAA |
| | | | CGCTG |
| AAAA | 157 | lolB.2 LF | TAATAAAGAATAGGATT |
| | 158 | lolB.2 LB | CAGACCATAAAGGAAA |

It will be appreciated that the primers listed in Tables 2 and 3 are designed to not only exhibit a high degree of specificity for the targeted DNA fragment, but also to enhance amplification speed. Indeed, the data provided herein supports that the primer sets provided herein render the LAMP assays hereof more rapid and more sensitive than conventional LAMP assays.

Any number of LAMP primer sets can be used in the same assay; for example, and without limitation, an assay can comprise a first LAMP primer set that targets a DNA fragment of rsmL, a second LAMP primer set that targets a DNA fragment of rsmC, and/or a third LAMP primer set that targets a DNA fragment of lktA (all genes identified to be specific to pathogen M. haemolytica). In another embodiment, for example, a first LAMP primer set can target a DNA fragment of lolA, a second LAMP primer set can target a DNA fragment of lolB, and/or a third LAMP primer set can target a DNA fragment of lppB (all genes identified to be specific to pathogen H. somni).

Additionally, the LAMP primer sets can comprise a combination of primer sets that each target DNA fragments of different pathogens. For example, a first primer set can target a DNA fragment of kmt1, ompP1, or ompl6 (all identified to be unique to pathogen P. multocida), a second primer set can target a DNA fragment of rsmL, rsmC, or lktA (all identified to be unique to pathogen M haemolytica), and a third primer set can target a DNA fragment of lolA, lolB, or lppB (all identified to be unique to pathogen H. somni).

The results of the LAMP assays hereof can, in some embodiments, be seen with the naked eye. While conventional versions of LAMP assays require SYBR Green staining for signal detection (which necessitates opening the tube after thermal incubation) the LAMP assays hereof can be performed with a turbidimeter (e.g., a Loopamp real-time turbidimeter) to detect a positive signal. A turbidimeter measures the relative clarity of the sample and does not require opening the tube, which reduces the risk of environmental diffusion and cross-contamination during gene amplification.

In certain embodiments, magnesium pyrophosphate produced during the reaction can be observed as a white precipitate or added indicators (e.g., calcein, magnesium-based indicators, or hydroxynaphthol blue) can be used to signal a positive reaction or an indicative pH change.

In certain embodiments, the LAMP assays hereof can be coupled with or include indicators (e.g., colorimetric reagents or indicators) to allow for visual inspection of assay results without opening the reaction tube. Such assay results can provide a visual result that corresponds to the presence or absence of the targeted pathogen for BRD in the sample. In some cases, the visual result is color-coded and/or colorimetric, and in other cases the result can be a letter, number, word, symbol, lines, or other representation indicative of the presence or absence of the targeted pathogen for BRD. For example, if one of the LAMP primer sets is targeted to a DNA fragment unique to P. multocida, if P. multocida is present within the sample, the LAMP primer set will identify and amplify that DNA fragment. Where the assay further comprises an indicator associated with each LAMP primer set, the indicator associated with the P. multocida primer set will be easily detectable in the results.

Fluorescence can also be employed to facilitate signal detection. In at least one embodiment, the LAMP assays hereof further comprise fluorescent dye in the reagents mix for assay or a fluorescent tag coupled with the primers themselves. Fluorescent data/intensities can thereafter be collected (using thermocyclers or a fluorometer, for example) and analyzed. In the above non-limiting example where a loop primer is directed to a unique DNA fragment associated with P. multocida, a particular fluorescent indicator can be coupled with such primer so that visualization of the fluorescence of that particular fluorescent indicator is indicative of the sample being positive for P. multocida.

As noted above, colorimetric reagents can be coupled with the primer set(s) of the LAMP assays described herein. In certain embodiments, the colorimetric agent is pH sensitive (e.g., phenol red). While specific embodiments and examples are provided herein, it will be appreciated that any colorimetric reagent sensitive to pH or magnesium can be employed.

Where multiple primer sets are used in the same assay, and each primer set is directed to a different pathogen, indicators can be used to easily identify in the visual results which pathogen(s) is present in the sample. In at least one embodiment, for example, the first primer set can be labeled (at their 5′-ends, for example) with a stable, fluorescent material of a first intensity, the second primer set can be labeled with a stable, fluorescent material of a second intensity, and the third primer set can be labeled with a stable, fluorescent material of a third intensity using methods commonly known in the relevant arts. When the relevant primer set anneals to a complementary target amplicon (i.e. the DNA fragment of the targeted pathogen), the 5′→3′ exonucleolytic activity of DNA polymerase detaches the label from the primer, which results in an enhanced fluorescence signal at the intensity of the fluorescent material used for the primer set with which there was a match. Accordingly, assessment of the resulting intensity can identify which pathogen is present within the sample. While fluorescent indicators are described above, it will be appreciated that any type of indicators can be used with the novel assays of the present disclosure, including other indicators now known or hereinafter developed.

Additionally, certain embodiments of the LAMP assays can optionally utilize a fluorescent reader, an ultraviolet light reader, and/or a camera for signal detection and/or the display of assay results (e.g., where indicators are used). In these embodiments, the visual results may be color-metric and/or digitally provided, such as, for example, through a wireless device, laptop computer, or cell phone and may utilize WiFi, Bluetooth, or cellular data.

The LAMP assays (and primer sets thereof) can detect the targeted pathogenic DNA fragments in various sample types and, in certain embodiments, does not require that such samples be processed prior to running the assay. For example, a sample can comprise a simple water sample or an unprocessed bovine nasal sample (e.g., obtained via a nasal swab). The ability to use unprocessed samples is advantageous for several reasons, at least one of which being that the assay translates easily to field use due to the ease of incubation. In certain embodiments, the samples, once collected, can be housed in a tube or vial containing a transport medium suitable for the collection, transport and/or handling of the specimen. For example, and without limitation, the transport medium can be liquid amies transport media.

Kits for testing one or more samples are also provided. Such diagnostic kits can be configured for field use such as, for example, in a feed lot or on-site at another type of cattle operation. Accordingly, the kits can be portable and capable of use in a non-laboratory setting.

In at least one embodiment, the diagnostic kits hereof comprise a molecular diagnostic assay comprising one or more of the LAMP primer sets described herein. In certain embodiments, such assays comprise one or more of the LAMP primer sets that comprise one or more primers of any of SEQ ID NOS. 1-6, 25-30, 55-62, 99-110, and 147-158.

The kit can further comprise at least one swab for obtaining a sample from a subject (e.g., a bovine) and/or a vial or other container for receiving the at least one swab after a sample is collected. In at least one exemplary embodiment, the container can be used as the incubation environment for the collected sample and one or more LAMP primer sets (i.e. where the amplification reaction is performed on the collected sample). Accordingly, the container can contain a transport media or the like as is known in the art, and/or any additional reagents that are useful in facilitating the DNA amplification reaction and/or visualizing the results thereof. For example, in at least one embodiment, UDG/UTG can be added to the media within the container to degrade leftover amplicons present therein after amplification of the targeted DNA.

In at least one embodiment, the container is sealable and is at least partially transparent such that visual results present within the container can be visualized without opening the container itself.

The assay of each kit can further comprise an indicator associated with each LAMP primer set. As described above, the LAMP primer sets can be configured to include the indicator (e.g., a fluorescent indicator coupled with an end of each primer) or the indicator can be added to the media housed by the container.

In certain embodiments, the indicator of each kit comprises a colorimetric reagent. For example, in certain embodiments, one or more of the LAMP primer sets can be coupled with a colorimetric reagent that is pH sensitive or magnesium sensitive. In certain embodiments, the colorimetric agent is phenol red.

The kit can further comprise a heating element to initiate amplification of the targeted DNA fragment when the at least one LAMP primer set and the sample are combined, for example, in the container. In certain embodiments, the heating element is a water bath. The kit can also, optionally, comprise a fluorescent reader, an ultraviolet light reader, or a camera to provide color metric result data indicative of the presence or absence of a targeted pathogen in the sample.

Methods for identifying a pathogen associated with BRD in a sample (and treating the same) are also provided. In at least one embodiment, a method for identification of a pathogen associated with BRD in a sample and treatment thereof comprises: providing at least one LAMP primer set that targets a DNA fragment of a targeted pathogen associated with a BRD in a sample; obtaining a sample from a subject; combining the sample and the at least one LAMP primer set into a mixture; heating the combination to initiate amplification of the targeted DNA fragment; and detecting a visual result in the heated combination indicative of the presence or absence of the targeted pathogen for BRD in the sample. In certain embodiments, of the visual result indicates the presence of at least one pathogen BRD present in the sample, the method further comprises identifying the type of pathogen present. In at least one embodiment, if the visual result is indicative of the presence of the targeted pathogen in the sample, the method further comprises treating the subject for the targeted pathogen.

As used herein, a “subject” is a mammal, preferably a bovine mammal, but it can also be a human or non-human animal (including, without limitation, a laboratory, an agricultural, a domestic, or a wild animal). Thus, the assays and methods described herein are applicable to both human and veterinary disease and applications. In certain embodiments, subj ects that can be addressed using the methods hereof include subjects identified or selected as having or being at risk for having BRD. Such identification and/or selection can be made by clinical or diagnostic evaluation.

At least one LAMP primer set can be any of the LAMP primer sets described herein. In at least one embodiment, for example, the at least one LAMP primer sets can be one or more primer sets A-AAAA identified above in Tables 2 and 3.

In at least one illustrative embodiment, the sample comprises a bovine nasal swab sample; however, any sample capable of providing a medium sufficient to detect the targeted pathogen(s) using the methods hereof can be used.

The methods hereof can be performed within a single container that need not be opened once the sample is placed therein. In at least one embodiment, the container includes (a) the desired LAMP primer set(s), each targeting a DNA fragment of a targeted pathogen associated with BRD; (b) a media to facilitate the storage and/or amplification reaction (e.g., water or liquid amies transport media), one or more indicators (either coupled with each primer set or added to the media), and/or (c) any additional reagents desired. Accordingly, in at least one embodiment, the sample is collected from the subject (e.g., a nasopharyngeal or nasal swab is used to collect a sample from a cow's nasal cavity) and placed within the container that already houses the assay and associated reagents. The container can then be sealed (e.g., via a screw cap or the like) and the container and its contents heated to initiate the amplification reaction.

Detecting the visual result produced by the method can be performed using any of the modalities described above. In certain embodiments, the visual results can be seen with the naked eye (without the use of additional instruments). In other embodiments, the assay further comprises one or more indicators associated with each set of loop primers such that detection of a particular indicator is indicative of the associated pathogen being present within the sample. Accordingly, the methods can additionally comprise using a turbidimeter to measure the relative clarity of the heated combination, and/or a fluorescent reader (e.g., a fluorometer), an ultraviolet light reader, or camera to analyze color metric data in the visual result.

These LAMP assay methods are advantageous over conventional methods because accurate results are more quickly produced, and the assays hereof are more sensitive and more specific than conventional methods. In certain embodiments, the methods hereof can provide a visual result indicative of the presence or absence of the targeted pathogen(s) within 45 minutes of initiating the heating step (e.g., initiating reaction of the primers with the sample). Further, such reactions can be conducted between about 60-65° C., which is well outside of ambient field temperatures. Importantly, the primers sets of the present disclosure have at least a 97% accuracy (96% sensitivity and 98% specificity), which is a significant improvement over conventional LAMP primers.

A previous limitation of LAMP assays for pathogen screening has been the occurrence of false positives either due to poor reagent handling or carryover contamination from previous experiments. With the present assay, methods, and kits hereof, this concern can be minimized by employing multiple spaces for reaction preparation, pre-aliquoting the required reagents in each container to reduce contamination losses, and/or adding UDG and dUTP to degrade leftover amplicons in incubation environments (i.e., the containers).

Unless defined otherwise, all technical and scientific terms used herein have the same meaning as commonly understood by one of skill in the chemical and biological arts. Although any methods and materials similar to or equivalent to those described herein can be used in the practice or testing of the subject of the present application, the preferred methods and materials are described herein. Additionally, as used in this specification and the appended claims, the singular forms “a”, “an” and “the” include plural referents unless the content clearly dictates otherwise.

When ranges are used herein, all combinations and sub-combinations of ranges and specific embodiments therein are intended to be included.

Additionally, the term “about,” when referring to a number or a numerical value or range (including, for example, whole numbers, fractions, and percentages), means that the number or numerical range referred to is an approximation within experimental variability (or within statistical experimental error) and thus the numerical value or range can vary between 1% and 15% of the stated number or numerical range (e.g., +/−5% to 15% of the recited value) provided that one of ordinary skill in the art would consider equivalent to the recited value (e.g., having the same function or result). The term “comprising” (and related terms such as “comprise” or “comprises” or “having” or “including”) is not intended to exclude that in other certain embodiments, for example, an embodiment of any compound, composition of matter, composition, method, or process, or the like, described herein, may “consist of” or “consist essentially of” the described features. The term “substantially” can allow for a degree of variability in a value or range, for example, within 90%, within 95%, or within 99% of a stated value or of a stated limit of a range.

Additionally, in describing representative embodiments, a method and/or process may have been presented as a particular sequence of steps. To the extent that the method or process does not rely on the particular order of steps set forth herein, the method or process should not be limited to the particular sequence of steps described. As one of ordinary skill in the art would appreciate, other sequences of steps may be possible. Therefore, the particular order of the steps disclosed herein should not be construed as limitations on the claims. In addition, the claims directed to a method and/or process should not be limited to the performance of their steps in the order written, and one skilled in the art can readily appreciate that the sequences may be varied and still remain within the spirit and scope of the present disclosure.

It is therefore intended that this description and the appended claims will encompass, all modifications and changes apparent to those of ordinary skill in the art based on this disclosure.

### EXAMPLES

The following examples illustrate certain specific embodiments of the present disclosure and are not meant to limit the scope of the claimed invention in any way.

### Example 1

### Design of LAMP Primers for BRD Pathogens

Published literature was investigated for highly conserved genes present in individual pathogens associated with bovine respiratory disease complex (BRD) and candidate gene sequences were run through the Basic Local Alignment Search Tool (BLAST) of the National Center for Biotechnology Information (NCBI). Any species-specific genes that had less than or equal to 50% similarity with other pathogen genomes were considered unique gene targets and used as template sequences for LAMP primer design (listed in Table 1). Additionally, multiple genomes of a single BRD pathogen were aligned using BLAST, and gene sequences that were conserved amongst alignments were manually determined. Chosen genes were then compared to genomes of other pathogens to ensure species-specificity by using the previously described criteria.

Three unique gene targets were chosen for each BRD pathogen to verify target pathogen identification for final assay. Three unique primer sets were designed for each gene target to conduct preliminary screening and optimize reaction performance.

All LAMP primer sets were generated using the publicly available Primer Explorer V5 software. Primer sets that met the following criteria were selected for initial screening: 1) spanned less than or equal to 200 bp of a target gene sequence, 2) had loop primers with a length of 18-21 base pairs (bp), and 3) had dG values of less than or equal to −4.0 kcal/mol for i) 3′ end of F2, ii) 5′ of F1c, iii) 3′ of B2, and iv) 5′ of B1c. For each gene target, a total of 3 unique LAMP primer sets were designed (see Table 3).

### Example 2

### Bacterial Strain and Complex Sample Collection

Pure isolates of Pasteurella multocida, Mannheimia haemolytica, and Histophilus somni were acquired in the form of glycerol stocks from the Indiana Animal Disease Diagnostic Laboratory (ADDL) at Purdue University (West Lafayette, IN). Nasal swabs were collected from 45 healthy heifers at the Purdue Animal Sciences Research and Education Center Beef Unit (PACUC #1906001911) using rayon tipped polyester swabs with liquid amies transport media (BD 220146). All nasal samples were pooled, vortexed until homogenous, and aliquoted for use as a complex substrate for cross-reactivity studies.

### Example 3

### Bacterial DNA Isolation

P. multocida, and M. haemolytica isolates were streaked on tryptic soy agar plates supplemented with defibrinated sheep blood (blood agar) and incubated aerobically at 37° C. for 16-18 hours. Single, isolated colonies of P. multocida and M. haemolytica were picked from plates, inoculated into brain-heart infusion (BHI) broth, and incubated aerobically at 37° C. for 16-18 hours.

H. somni isolates were similarly streaked on blood agar plates, stored in BD GasPak™ EZ container systems (BD 260672) with BD BBL™ CO₂ gas generators (BD 260679), and incubated in a 5% CO₂ atmosphere at 37° C. for 2-3 days or until sufficient colony growth was present.

H. somni colonies were inoculated into tryptic soy broth (TSB), stored in the previously mentioned BD GasPak™ EZ container system with the CO₂ gas generators and incubated with 5% CO₂ at 37° C. for 2-3 days.

Genomic DNA of all bacterial isolates were extracted by taking 1-2 mL of saturated liquid culture and processing them through the PureLink™ Genomic DNA Mini Kit (Invitrogen K182002, Invitrogen, Waltham, MA) with a final eluted volume of 30 μL. Final DNA concentrations (ng/μL) of eluted extracts were measured using the Quant-iT PicoGreen dsDNA Assay Kit (Invitrogen P11496, Invitrogen, Waltham, MA).

### Example 4

### Bacterial DNA Verification

Polymerase chain reaction (PCR) reactions were conducted on extracts using gene-specific target primers and run on 2% agarose gels to confirm bacterial genome identity. Gels were 2% w/v agarose and were run in a horizontal electrophoresis chamber in Tris Borate EDTA (TBE) for 60 minutes at a voltage of 80 V. The bands were seen at expected locations as confirmed by a 1 kb ladder (gel images not shown).

### Example 5

### Evaluation of Primer Performance through Quantitative LAMP Assay (qLAMP)

Three genes were selected for targeting each bacterial target (i.e. the targeted pathogen) (see FIG. 1). Of all designed LAMP primer sets listed in Table 3 above, those that were optimal for the targeted genes was determined by first designing multiple primer sets per gene and then characterizing their performance. Initial screening of primer sets was carried out to identify sets that were able to amplify genomic DNA (gDNA) from a target pathogen, while maintaining little to no amplification of other non-targeted pathogens or negative samples.

LAMP reactions were conducted by following manufacturer instructions of the Warmstart® LAMP Kit (DNA & RNA) (E1700L New England Biolabs, Ipswich, MA). 25 μL reactions were comprised of 12.5 μL of Warsmtart® LAMP 2× Master Mix (40 mM Tris-HCl, 20 mM (NH₄)₂SO₄, 100 mM KCl, 16 mM MgSO₄, 2.8 mM dNTPs, 0.28 μM dUTP, 0.64 U/μL Warmstart Bst 2.0 DNA polymerase, 0.6 U/μL Warmstart Reverse Transcriptase [RTx], 4×10−4 U/μL Antarctic Thermolabile UDG, 0.2% Tween 20, pH 8.8@25° C.), 2.5 μL of a 10× LAMP primer mixture (2 uM F3, 2 uM B3, 4 uM LF, 4 uM LB, 16 uM FIP, 16 uM BIP) 5 μL of a 1:101 dilution of the included LAMP fluorescent dye, and 5 μL of the template DNA containing solution.

Antarctic Thermolabile UDG and dUTP were added to the LAMP reaction mixture for detection limit and complex cross-reactivity studies to minimize carryover contamination during assay preparation. In-house validation experiments confirmed that the addition of UDG/UTP did not affect reaction performance. Unless specified, the final concentration of template DNA for qLAMP reactions was 1 ng/reaction.

Reactions were pipetted into wells of white 96-well full-skirted PCR plates (AB-0800W Thermo Fisher Scientific. Waltham, MA). Wells were sealed with VersiCap Mat Cap Strips (AB1820 Thermo Fisher Scientific, Waltham, MA) and were inserted into either a CFX96 Touch Real Time PCR Detection System (Bio Rad) or a qTOWER³ G (Analytik Jena, Jena, Germany) for real-time fluorescent measurement. Reaction plates were incubated at 65° C. for 1 hour with fluorescent scans taken using the FAM/SYBR Green I filter every minute. A ramp rate of 6° C./s and 8° C./s was used on the CFX96 and qTOWER³ G respectively. A ramp rate of 0.1° C./s was used on the qTOWER³ G for detection limit and complex reactivity experiments to improve the overall detection limit of the LAMP reactions.

To minimize false positives due to amplicon aerosol contamination, LAMP reaction mixture preparation, template DNA loading, and reaction incubation/measurements were conducted in separate lab spaces. RNase AWAY® Surface Decontaminant (14-754-34 Thermo Fisher Scientific, Waltham, MA) was thoroughly applied to all working surfaces, reagent containers, pipettes, and lab gloves before and after each lab space operation, and the same were wiped completely with Kimwipes to prevent residue formation. Care was taken in the following three ways: i) plate agitation was minimized during reaction preparation and DNA loading, ii) cap strips to wells were securely depressed before and after assay steps, and iii) plates were wrapped with aluminum foil (cleaned with RNase AWAY) for transport between lab spaces.

Additionally, as described below in Examples 6 and 7, all designed primers (see Table 3 above) were run with gDNA in water to test for primer dimerization (i.e. early amplification of negative controls) or cross-reactivity with off-target DNA. Moreover, primer sets with faster reaction speeds and more consistent trends of amplification (i.e. smaller standard deviation (SD)) were given higher priority for selection.

Performance characteristics of all designed primer sets selected for screening are shown in Table 4, with the highlighted rows indicating those primer sets with optimal reaction features that were chosen for further assay development based on the highest total score.

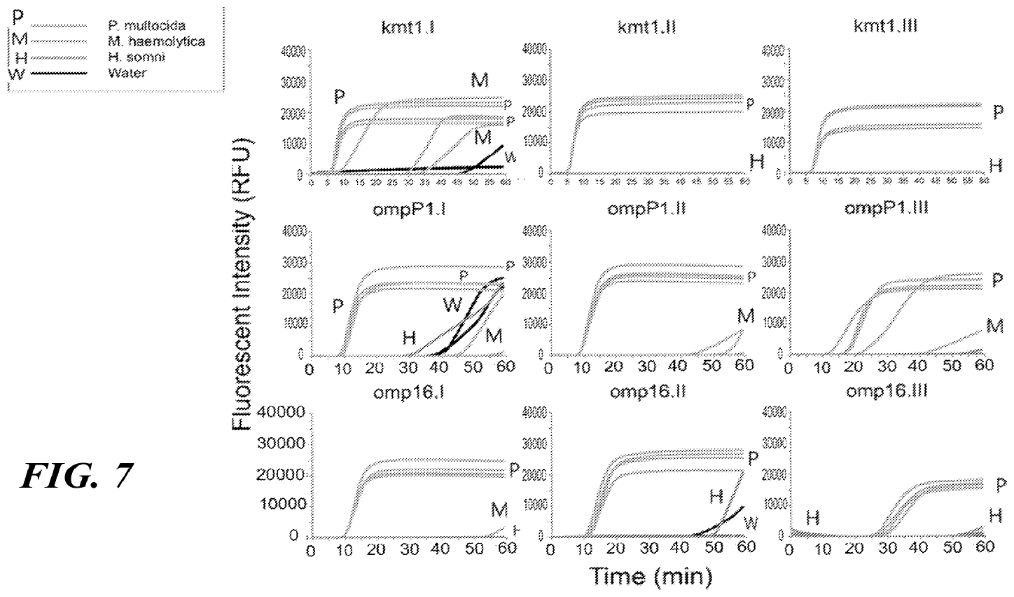

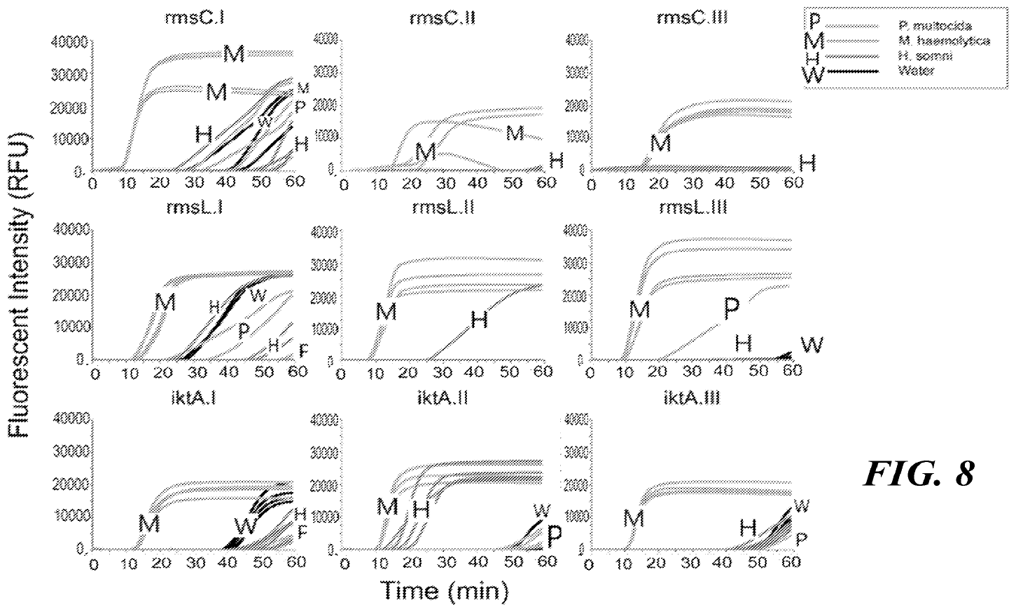

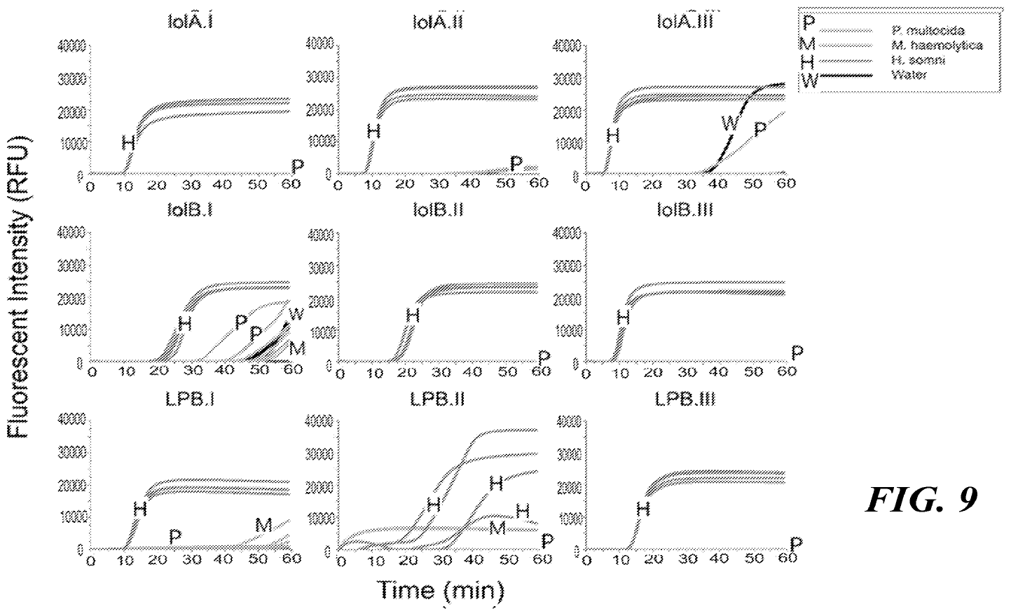

The results of the screening (see at least Table 4 and FIGS. 7-9) indicate that the following primer sets were considered optimal (Table 2 for sequences): 1) kmt1.2 (SEQ ID NOS: 1-6), ompP1.2 (SEQ ID NOS: 7-12), and omp16.1 (SEQ ID NOS: 13-18) for P. multocida, ii) rsmC.3 (SEQ ID NOS: 31-36), rsmL.2 (SEQ ID NOS: 25-30) and lktA.3 (SEQ ID NOS: 19-24) for M. haemolytica, and iii) lolA.2 (SEQ ID NOS: 43-48), lolB.3 (SEQ ID NOS: 49-54), and lppB.3 (SEQ ID NOS: 39-42) for H. somni.

### Example 6

### Primer Screening

Fluorescent data collected from real-time thermal cyclers were exported as excel worksheets (.xlsx) and manipulated in Excel or custom MathWorks MATLAB® scripts. See the Tables and Figures herein.

Response time was determined by taking the timepoint at which 90% of the maximum reaction intensity occurs. False positive reactions were defined as reactions with negative/non-target controls that had fluorescent intensities higher than 20% of the maximum reaction intensity. Individual performance metric data for all screened primer sets were normalized and multiplied by numerical weights to generate metric scores. All metric scores for a single primer set were then summed to create a total performance score. Any primer sets that produced non-target amplification in less than 30 minutes were automatically rejected and given a total performance score of 0.

### Example 7

### Cross-Isolate/Reactivity of LAMP Assays Across Multiple Sample Isolates

The specificity of certain identified primer sets was tested by comparing the amplification results of the LAMP primers with off-target DNA.

For cross-isolate data, fluorescent intensities were extracted for the 30-minute time-point for all primer set reaction replicates and arranged in a table ordered by isolate. Table values were converted to a heat map and formatted using OriginLab® OriginPro (OriginLab Corporation, Northampton, MA), to visually display amplification differences between primer sets for all isolates.

For cross-reactivity data, fluorescent intensities were extracted for the 45-minute time-point for all primer set reaction replicates (because these samples have a lower concentration of spiked-in DNA) and arranged in a similar manner to cross-isolate data.

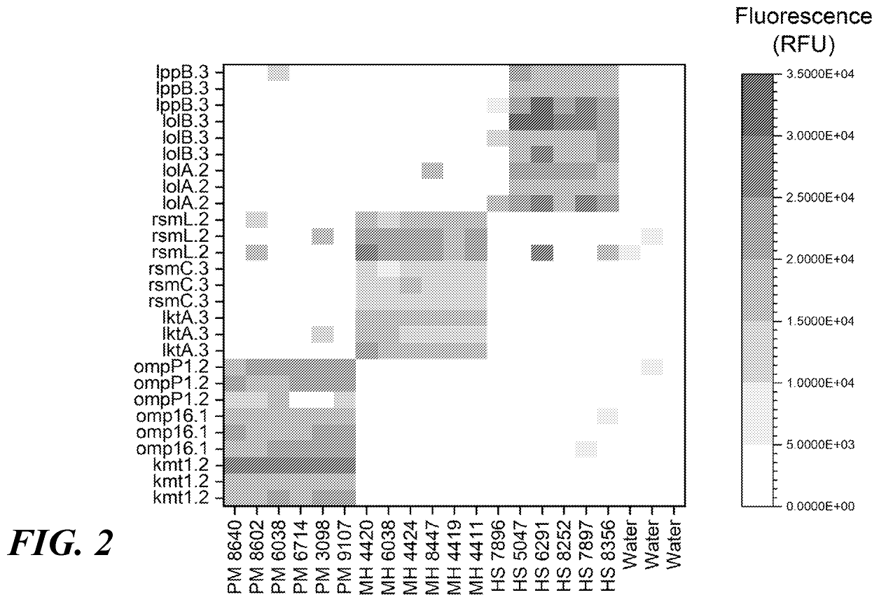

As shown in FIG. 2, most of the primer sets amplified the target pathogenic DNA and did not amplify off-target pathogenic DNA.

### EXAMPLE 8

### Receiver Operator Characteristic Curve

Receiver-operator characteristic curves were generated by comparing formatted cross-isolate data to a predefined threshold via binary classifications to assess positive vs negative reactions. Thresholds were defined as a percentage of the maximum fluorescent intensity of the data set. Various thresholds (0%-100%) were tested and the equations below were used to calculate true positive rate and false positive rate for each threshold classification. Sensitivity and specificity of the LAMP assay to cross isolate data (see FIG. 2) was defined as the true positive rate and 1-false positive rate for the threshold value that created the greatest difference in sensitivity between the receiver operating characteristic (ROC) curve and the random chance line. Accuracy was determining by taking the area under the ROC curve.

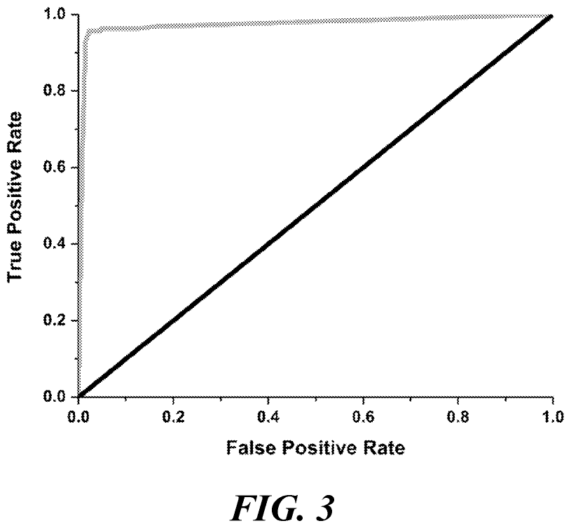

The resulting receiver-operating characteristic curve data is shown in FIG. 3. By generating a receiver-operating characteristic curve based off the cross-reactivity data (Example 7; FIG. 2), it was determined that the primers had 97% accuracy (96% sensitivity and 98% specificity) when using a fluorescent threshold of 28% of the maximum reported intensity. Using different isolates of the same species helped to check for cross-reactivity in case there were strain-specific genetic differences that could influence reaction performance. Since most isolates showed consistent amplification results with their own species, the LAMP primer sets of the present disclosure functioned as expected.

One exception was the H. somni isolate 7896 that did not amplify reliably with any of the inventive LAMP primer sets. Further sequencing and genome annotation using RAST revealed that this isolate was putatively identified as Staphylococcus hominis, which has no significant similarity with H. somni and whose genomic DNA would not be expected to amplify with the present H. somni primer sets. As such, it is likely that this isolate was mislabeled or contaminated during handling.

### Example 9

### Determination of Assay Detection Limits in Simple and Complex Samples

Optimal primer sets were characterized using LAMP reactions of decreasing concentrations of target gDNA template to assess limits of detection (LoD). “LoD” or “LOD” means the lowest concentration at which 3/3 replicates show amplification. LoDs were predominantly 1) 10³ copies/reaction in water samples, and 2) 10⁴ copies/reaction in liquid amies samples, with the order of magnitude difference between the two media types likely due to the LAMP reaction composition being altered by the increased salt concentrations present in the liquid amies, which can negatively impact reaction sensitivity.

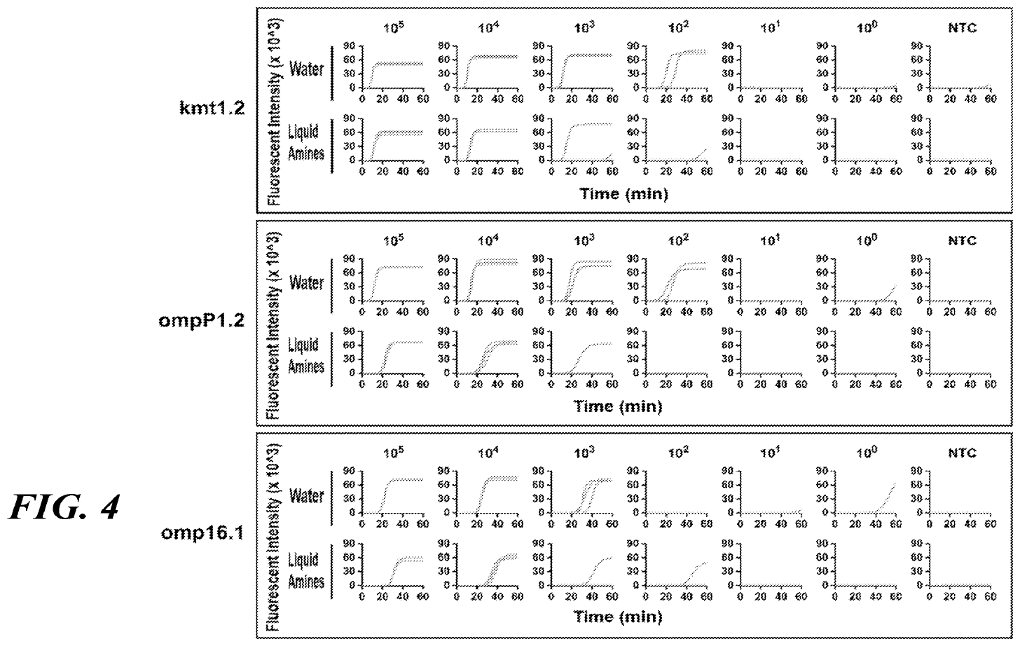

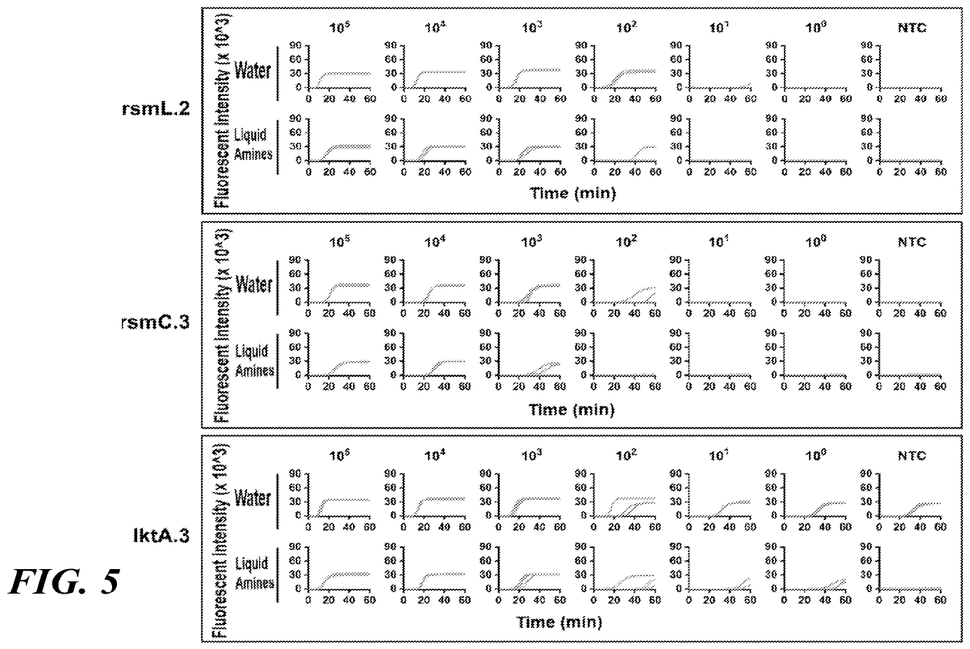

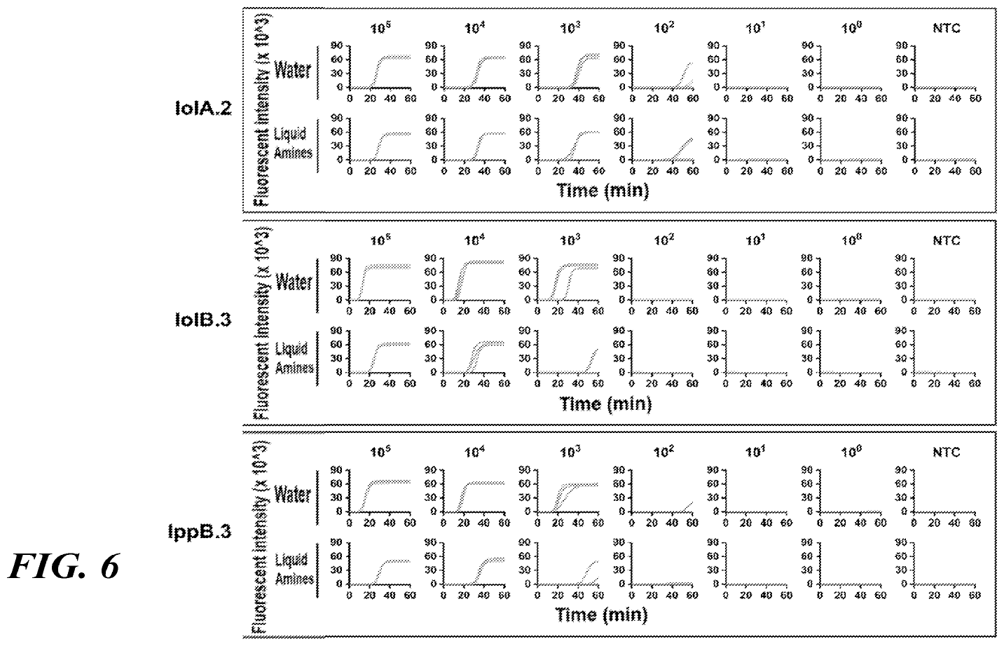

LoD experiments were conducted on target gDNA suspended in water and liquid amies separately to determine inhibitory effects on reaction performance. The performance of some embodiments of primer sets hereof is highlighted in FIG. 1. Further, as shown in FIGS. 4-6, as the DNA concentration decreased, an associative increase in response time of all primer sets was observed.

Fluorescent intensities were extracted for the 45-minute time-point for all primer set reactions. Intensities were divided by the maximum intensity of the LoD data set and multiplied by 100 to represent a percent amplification value. Any amplification values that were greater than the previously determined ROC threshold (percent amplification) were highlighted light blue and considered successful amplifications. The lowest DNA concentrations that had successful amplifications for all three replicates of a given primer set were classified as the LoD for the primer set.

### Example 10

### Preparation of Bacterial Isolates and Mucus Sample Collection for Colorimetric Studies

Glycerol stocks of P. multocida, M haemolytica, and H. somni isolates were obtained from Purdue University's ADDL as described in Example 2 above. These isolates were originally cultured by ADDL as a part of routine diagnostic testing from lung/nasopharyngeal sample submissions and identified using matrix-assisted laser desorption/ionization time-of-flight mass spectrometry (MALDI-TOF MS) analysis.

Mucus samples were obtained from steers (n=5) approximately 12 months of age and 600 lbs. in weight that had not been given antibiotics for at least 100 days at a feedlot in Indiana (Purdue Animal Care and Use Committee Approval #1906001911) using rayon-tipped sterile double swabs designed for general specimen laboratory use (BD 220135, Becton, Dickinson, and Company, Franklin Lakes, NJ). Each animal was restrained in a livestock handling chute and the animal head was restrained to minimize movement. The nostrils were wiped with paper towels to remove excess mucus. One double swab was inserted into both nostrils sequentially at a depth of approximately 5 cm. The swabs were then swirled in tubes with 200 μL of DNA-free water and aliquoted for use in LAMP reactions for both on-farm and in-lab settings.

All nasal samples were pooled, vortexed until homogenous, and aliquoted for use as a complex substrate for cross-reactivity studies. The bacterial DNA was then isolated and quantified pursuant to the protocols set forth in Example 3 above.

### Example 11

### Colorimetric Quantitative LAMP Assay (qLAMP)

The colorimetric assay was conducted by modifying the procedure described in the above examples. Specifically, in the colorimetric assay, the New England Biolabs' Warmstart® Colorimetric LAMP 2× Master Mi was employed. The mix was coupled with Antartic Thermolabile uracil DNA glycosylase (UDG) and deoxyuridine triphosphate (dUTP) to minimize carryover contamination throughout the experiment. In-house validation experiments confirmed that UDG/dUTP did not affect reaction performance at the concentrations used.

The LAMP solution comprised 12.5 μL of this mix (40 mM Tris-HCL, 20 mM (NH₄)₂SO₄, 100 mM KCl, 16 mM MgSO₄, 2.8 mM dNTPs, 0.28 μM dUTP, 0.64 U/μL Warmstart® Reverse Transcriptase (RTx), 4×10⁴ U/μL Antarctic Thermolabile UDG, 200 mM Phenol red, 0.2% Tween 20, pH 8.8@25° C.) (Catalog #M1800L, New England Biolabs, Ipswich, MA), 2.5 μL of a 10× LAMP primer mixture (10× concentration: 2 μM B3, 4 μM LF, 4 μM LB, 16 μM forward inner primer (FIP), 16 μM backward inner primer (BIP)), 5 μL of DNA-free water, and 5 μL of DNA or mucus containing solution.

Reactions were pipetted into wells of clear 96-well FrameStar® skirted flat optical bottom PCR plates (Thomas Scientific, Swedesboro, NJ). Wells were sealed with adhesive PCR plate seals (Thermo Fisher Scientific, Waltham, MA) and inserted into a CLARIOstar Plus (BMG Labtech, Cary, NC), a multi-mode plate reader with temperature control, for real-time colorimetric measurement.

Spectra were collected from 350 to 750 nm with a step size of 5 nm for 60 cycles lasting approximately 60 seconds each. Reaction plates were incubated at 65° C. using the CLARIOstar Plus.

Each step in LAMP preparation (primer addition, template DNA loading, and reaction incubation/measurement) was conducted in separate lab spaces to minimize false positives due to amplicon aerosol contamination. To further reduce contamination, RNase AWAY™ Surface Decontaminant (Thermo Fisher Scientific, Waltham, MA) was thoroughly applied to all working surfaces and lab gloves before and after use and wiped completely with Kimwipes to prevent residue formation.

Absorbance measurements for each minute at 430 nm, 560 nm, and 620 nm wavelengths were extracted, and the data normalized using the formula of Equation (1):

$$Colorimetric absorbance ratio = \frac{Absorbance at 430 nm - Absorbance at 620 nm}{Absorbance at 520 nm - Absorbance at 620 nm} \qquad ( 1 )$$

The absorbance at 620 nm was used as a baseline, and the 430 nm and 520 nm wavelengths were used to mark the change in color of phenol red from red to yellow. The resulting ratios were plotted against time in Microsoft Excel.

### Example 12

### Colorimetric Threshold

A one-to-one mixture of pH 7.2 phosphate-buffered saline (PBS) (Thermo Fisher Scientific, Waltham, MA) and pH 8.5 Tris-HCL (Bio Basic, Amherst, NY) was prepared. Using 0.1 M NaOH and 0.1 M HCl, the solution was adjusted to range between pH 6.0 and 8.0 with increments of approximately 0.2 pH units. 5 μL of the buffer solution were added to 12.5 μL of Warmstart® Colorimetric LAMP 2× Master Mix (which contains phenol red) and 7.5 μL of DNA-free water. Each condition was added to a 96-well FrameStar® skirted flat optical bottom PCR plate in triplicate, sealed with a PCR film, and inserted into the CLARIOstar Plus to obtain measurements for three minutes. Data was analyzed as explained in Example 11 above and the values at each minute were averaged.

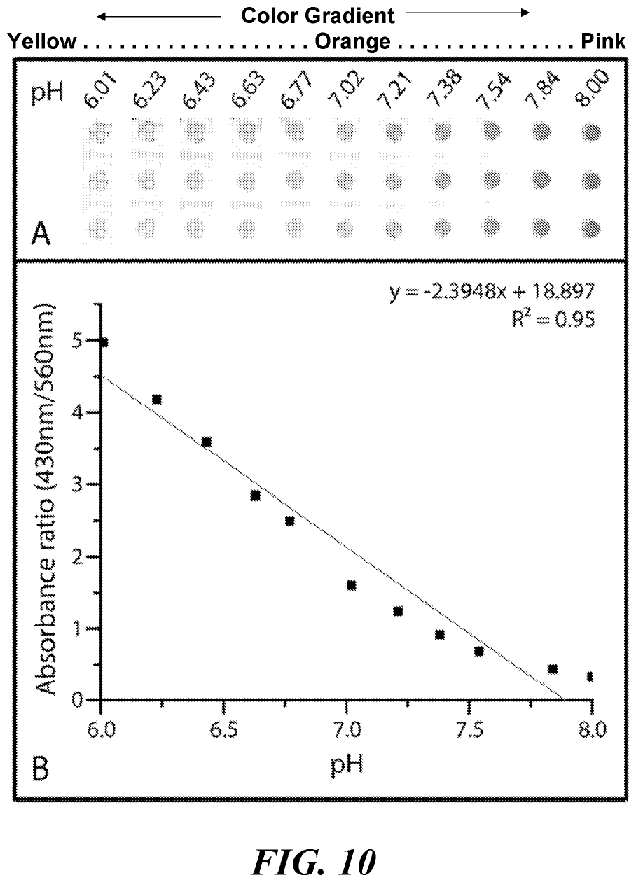

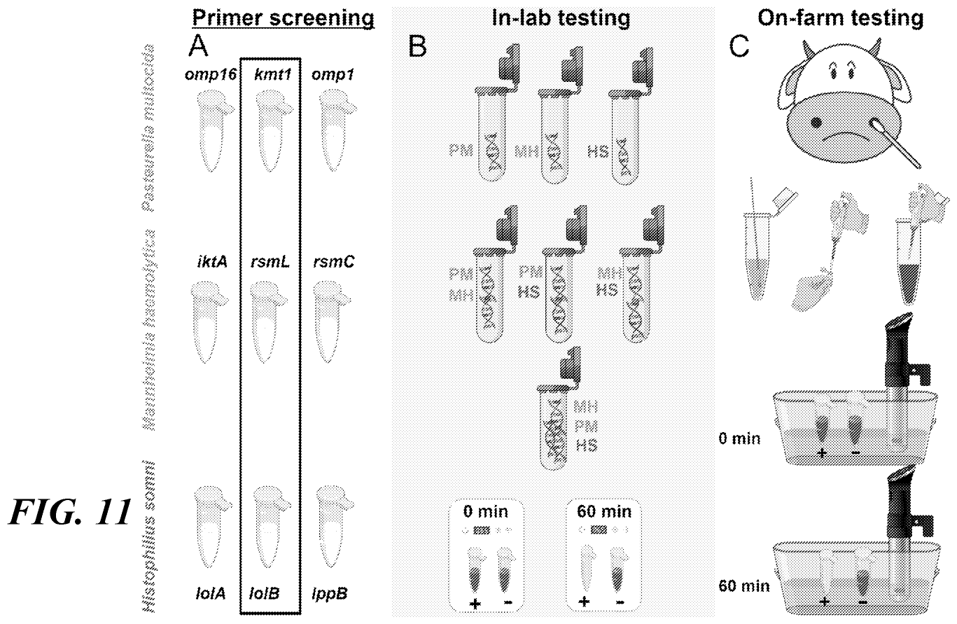

An image of the plate was taken using an Epson Perfection V800 Photo scanner (Amazon, Seattle, WA) with settings set to professional mode, 48-bit color image type, and 720 dpi resolution. As shown in FIG. 10A, LAMP reaction reagents were set to increasing pH to visualize color change. The colorimetric results (i.e. average colorimetric absorbance ratios from three cycles collected with the CLARIOstar Plus) where then plotted as shown in FIG. 10B.

3.0 was selected as the threshold for colorimetric absorbance ratio according to the color changes observed so that colorimetric absorbance ratios above the threshold were considered positive and colorimetric absorbance ratios below the threshold were considered negative. The image of the sample closest to the threshold value was processed using ImageJ to find the RGB values. Those RGB values were in turn used to calculate Hue Saturation Values (HSV) values. Perhaps more specifically, the color of the LAMP reaction at pH 6.63 (which is the closest to the 3.0 threshold of all samples tested) was used to set the Hue threshold at 35 since, at this point, the color change is distinctly different from the starting reaction color around pH 7.5-8.

The HSV threshold was used to determine positive (above HSV threshold) versus negative (below HSV threshold) results in other assays.

### Example 13

### Colorimetric Assay Primer Screening and LOD

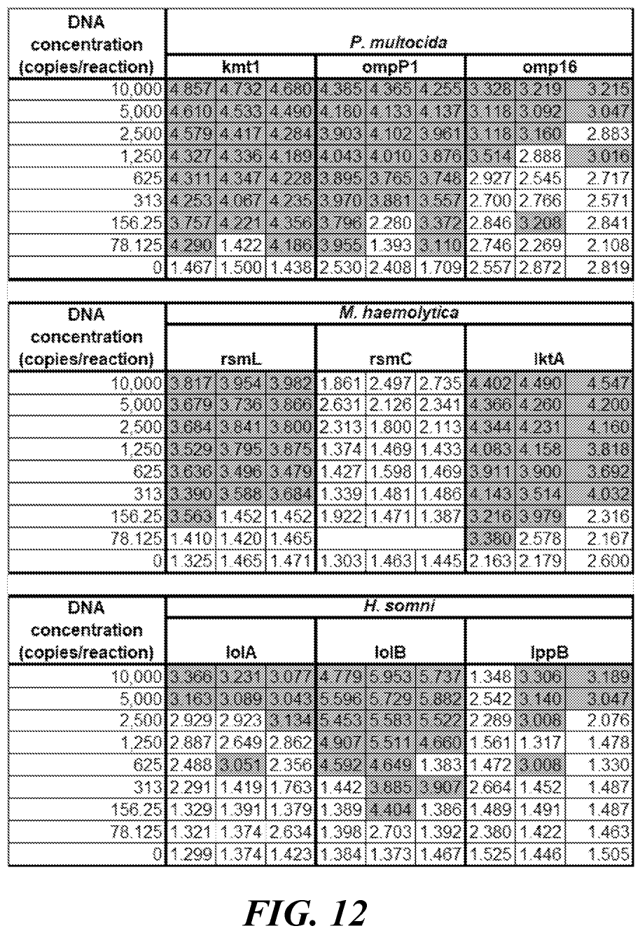

Various primers were screened through a LOD study to identify the best performing for each species of bacteria studied (see FIG. 11A).

Colorimetric absorbance ratios were obtained from qLAMP experiments using 2× DNA dilution factor (10 000—78.125 copies of DNA per reaction), noting that the fraction of copies was included simply to indicate the dilution factor and the actual number of copies in a reaction was rounded to the nearest whole number. All primer sets in Tables 2 and 3 (SEQ. NOS 1-158) (the “selected primers”) were tested and each concentration of the template included three replicates for each primer set. DNA-free water was used as a control (no-template control (NTC)).

Primer sets were scored by annotating the number of sufficient amplification reactions—defined as any replicate whose colorimetric absorbance ratio at 60 minutes was greater than 3.013 for each template concentration (including NTC) for each primer set. Any replicate that was deemed as sufficient amplification in NTC was designated as a false positive. Any missing data for an entire template concentration was set at a constant value equal to the maximum colorimetric absorbance ratio observed across all primer sets at all concentrations. In contrast, any missing data for any given time point was filled with the value of the previous time point.

Primer sets were scored by first calculating the maximum colorimetric absorbance ratio and reaction time for each replicate at each template concentration (excluding NTC) for a given primer set. The average and standard deviation of these values were then calculated for each template concentration for a given primer set. Reaction time was defined as the first time point at which the absorbance ratio was greater than 3.0. For each primer set, the average of each one of these four metrics (average and standard deviation of maximum intensity and reaction time) was calculated across all template concentrations to assign a primer set metric (e.g., primer set average maximum colorimetric absorbance ratio). The LOD for each concentration was then calculated as the minimum template concentration where all replicates sufficiently amplify and all replicates of template concentrations above this minimum template concentration also sufficiently amplify. For cases where all replicates for all tested template concentrations amplified, the LOD was set at the lowest non-zero template concentration if there were less than three false positives. If all NTC reactions amplified (i.e. three false positives) or no replicates amplified at any template concentration, the LOD was set at −1.

For the overall scoring of primer sets, ineligible primer sets (as designated by an LOD of −1) were automatically assigned an overall score of 0 and withdrawn from further scoring. All eligible primer sets were then assigned a weighted overall score, Sₖ, for a primer set k using the following expression:

$${S}_{k} = {w}_{\overline{I}} \cdot ( 1 - \frac{max ( \overline{I} ) - {I}_{k}}{Range ( \overline{I} )} ) + {\sum }_{n} {w}_{n} \cdot ( 1 - \frac{min ( n ) - {n}_{k}}{Range ( n )} )$$

$$n ∈ ( σ ( I ) , \overline{{t}_{rxn}} , σ ( {t}_{rxn} ) , LOD , FP )$$

$${w}_{n} = [ \begin{matrix}\begin{matrix}\begin{matrix}\begin{matrix}\begin{matrix}35 \\ 5\end{matrix} \\ 30\end{matrix} \\ 5\end{matrix} \\ 5\end{matrix} \\ 20\end{matrix} ] for \begin{matrix}\begin{matrix}\begin{matrix}\begin{matrix}\overline{I} \\ \frac{σ ( I )}{{t}_{rxn}}\end{matrix} \\ σ ( {t}_{rxn} )\end{matrix} \\ LOD\end{matrix} \\ FP\end{matrix}$$

where Ī, σ(I), t̅ᵣ̅ₓ̅ₙ̅, σ(tᵣₓₙ) LOD, FP is the set average maximum colorimetric absorbance ratio, set standard deviation of the maximum colorimetric absorbance ratio, set average reaction time, set standard deviation of the reaction time, set LOD, and the number of false positives for a given primer set, respectively. The range defined above is the maximum value minus the minimum value for a given set metric across all eligible primer sets. If the range for a given set metric was 0 (i.e. all primer sets had the same value), that set was given the full weight allotted for that set metric.

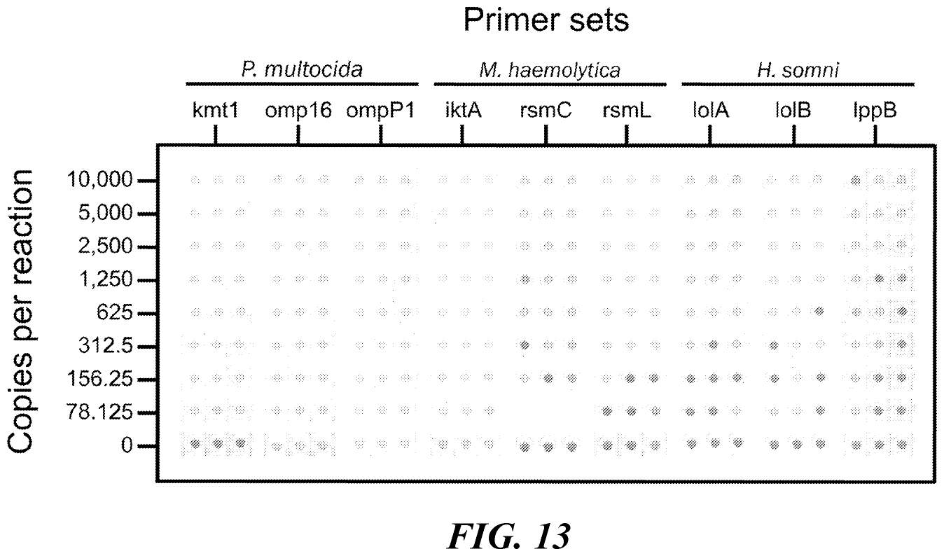

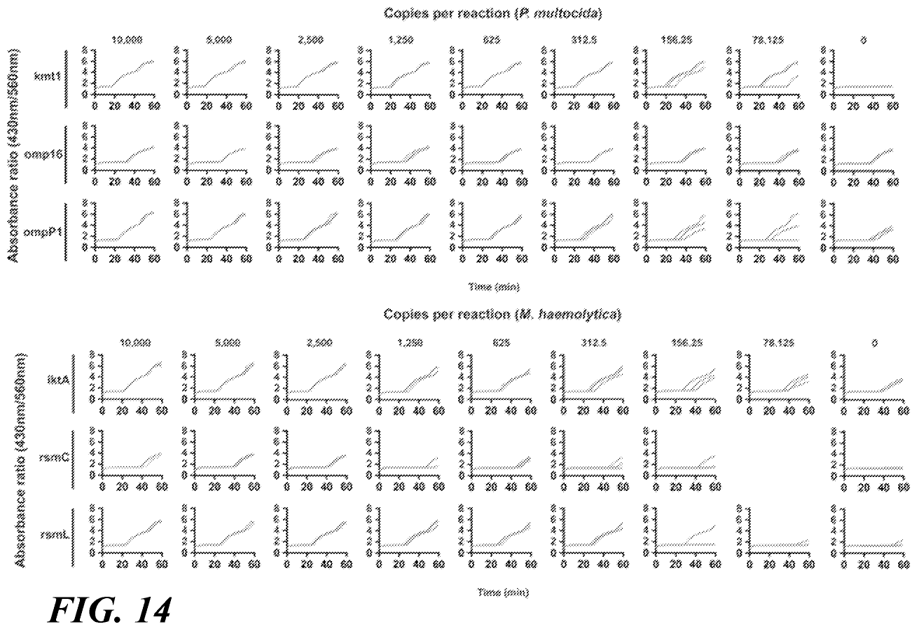

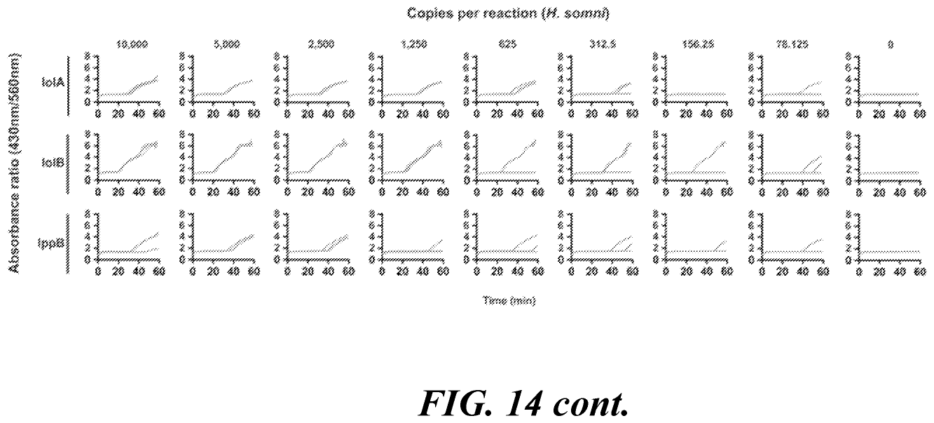

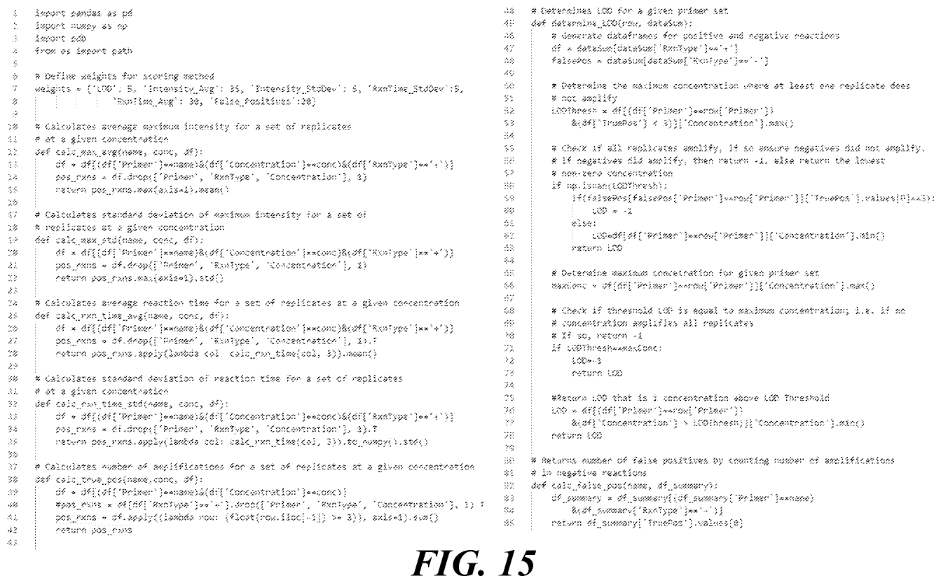

The selected primers were narrowed down further to one per bacterial target. Primers with the highest calculated scores obtained from LOD colorimetric assays (see FIGS. 12-14) were identified as the most-optimal primer sets to detect the bacteria of interest using a python script (see FIG. 15). The primers with the highest scores for the identified target pathogen were kmt1, rsmL, and lolB as highlighted in the data shown in Table 5 below. The worst LOD of the three selected primers, 1250 copies per reaction, was selected as the concentration used in later multiple isolate experiments. Images of the plates were taken at 0 and 60 minutes using the Epson Perfection V800 Photo scanner, as shown in FIG. 16.

### Example 14

### Combinatorial Study with One, Two, or Three Species Spiked into Water Using Phenol Red

Colorimetric qLAMP reactions with the selected primer sets were performed to determine assay performance with mixed bacterial samples (i.e. to determine their analytical sensitivity and specificity by examining their behavior with on-target and off-target DNA mixtures). In particular, several combinations of DNA were diluted in water and tested in a lab environment to study off-target behavior in pH-sensitive colorimetric reactions (see FIG. 11B).

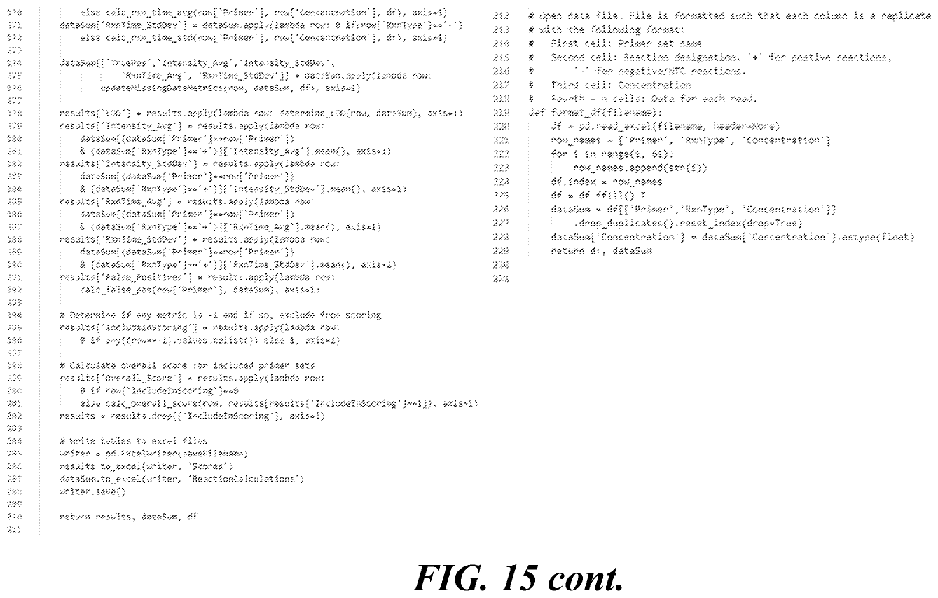

Colorimetric qLAMP assays were performed for 60 minutes using the previously determined LOD (1250 copies per reaction of gDNA) of one, two, and/or three of the following pathogens being tested in a reaction: P. multocida, M haemolytica, and H. somni. Pathogens were tested in pairs and all together to simulate the bacterial communities present in actual mucus samples. Each condition was repeated nine times in nine separate wells of 96-well plates. Images of the plates were taken at 0 and 60 minutes using the Epson Perfection V800 Photo scanner (see FIG. 17). Colorimetric absorbance ratios were calculated as explained above, and the resulting data were plotted against time (FIG. 18).

The kmt1 (PM) and lolB (HS) primer sets were able to amplify the corresponding genes regardless of the bacterial combination and showed minimal false-positive results. Rsml (MH) also showed a colorimetric difference in the presence versus the absence of M. haemolytica genes. However, it was not significant enough for the previously determined standards of 3.0 colorimetric absorbance threshold and 35 Hue value since some samples with target pathogenic DNA showed results below these values (see FIGS. 17 and 18).

The finalized data were also analyzed in ROC curves for each primer set (using the colorimetric threshold previously determined and assessing positive versus negative reactions for each primer set). The highest number obtained from subtracting the false positive rate (Equation 2) from the true positive rate (Equation 3) was selected as the time threshold for that specific primer.

$$False positive rate ( F P R ) = \frac{no . of false positives}{no . of false positives + no . of true negatives} \qquad ( 2 )$$

$$True positive rate ( T P R ) = \frac{no . of true positives}{no . of true positives + no . of false negatives} \qquad ( 3 )$$

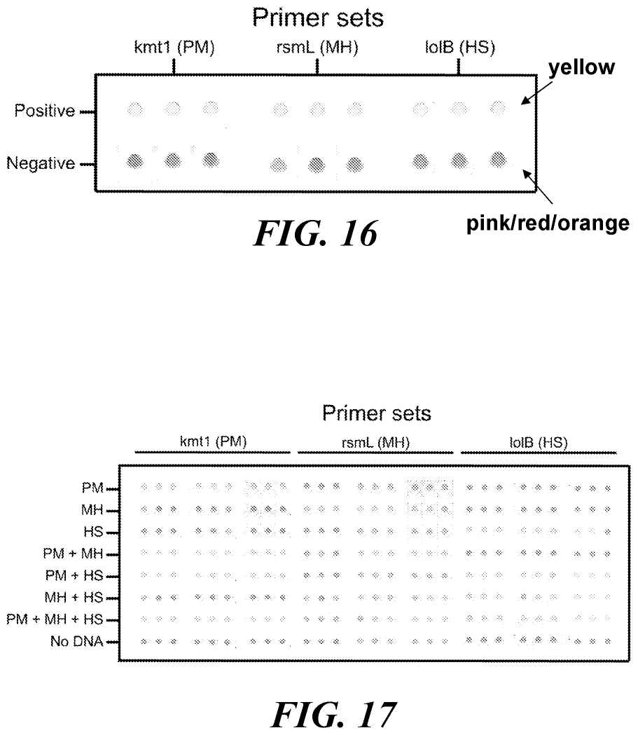

As shown in FIG. 19, the kmt1 and lolB primer sets showed nearly perfect curves (analytical sensitivity of 100% (kmt1) and 91.67% (lolB); analytical specificity of 100% (kmt1) and 100% (lolB), while rsmL did not. This suggests that rsmL does not work well (66.7% analytical sensitivity; 100% analytical specificity) and may benefit from redesign in future studies to improve analytical sensitivity.

These curves were also used to identify a time threshold for each of the primer sets by finding the time throughout the 60-minute period with the greatest difference between the true positive and false-positive rates (i.e. if a reaction requires more than this threshold to obtain a colorimetric absorbance ratio of 3.0, it is considered a negative result). The selected time thresholds were: 41 minutes (kmt1), 59 minutes (rsmL), and 54 minutes (lolB) (see Table 6).

TABLE 6
LAMP absorbance time thresholds with different
combinations of PM, MH, and HS gDNA.

| Time | | | | | | | | | | | | | |
|---|---|---|---|---|---|---|---|---|---|---|---|---|---|
| threshold | Pri- | | | | | | | | | | | | |
| (minutes) | mers | PM | | | MH | | | HS | | | PM + MH | | |
| 41 | kmt1 | 34 | 34 | 34 | 60 | 60 | 60 | 60 | 60 | 60 | 35 | 36 | 35 |
| | | 38 | 34 | 35 | 60 | 60 | 60 | 60 | 60 | 60 | 37 | 36 | 36 |
| | | 39 | 38 | 36 | 60 | 60 | 60 | 60 | 60 | 60 | 39 | 37 | 38 |
| 59 | rsmL | 60 | 60 | 60 | 56 | 42 | 45 | 60 | 60 | 60 | 60 | 44 | 43 |
| | | 60 | 60 | 60 | 59 | 50 | 56 | 60 | 60 | 60 | 60 | 49 | 44 |
| | | 60 | 60 | 60 | 60 | 51 | 60 | 60 | 60 | 60 | 60 | 59 | 49 |
| 54 | lolB | 60 | 60 | 60 | 60 | 60 | 60 | 37 | 39 | 41 | 60 | 60 | 60 |
| | | 60 | 60 | 60 | 60 | 60 | 60 | 41 | 39 | 53 | 60 | 60 | 60 |
| | | 60 | 60 | 60 | 60 | 60 | 60 | 46 | 40 | 60 | 60 | 60 | 60 |
| Time | | | | | | | | | | | | | |
| threshold | Pri- | | | | | | | PM + | | | | | |
| (minutes) | mers | PM + HS | | | MH + HS | | | MH + HS | | | NTC | | |
| 41 | kmt1 | 34 | 36 | 37 | 60 | 60 | 60 | 36 | 36 | 36 | 60 | 60 | 60 |
| | | 35 | 36 | 40 | 60 | 60 | 60 | 37 | 38 | 37 | 60 | 60 | 60 |
| | | 39 | 37 | 41 | 60 | 60 | 60 | 39 | 38 | 37 | 60 | 60 | 60 |
| 59 | rsmL | 60 | 60 | 60 | 60 | 55 | 43 | 60 | 44 | 42 | 60 | 60 | 60 |
| | | 60 | 60 | 60 | 60 | 60 | 45 | 60 | 58 | 45 | 60 | 60 | 60 |
| | | 60 | 60 | 60 | 60 | 60 | 60 | 60 | 60 | 54 | 60 | 60 | 60 |
| 54 | lolB | 37 | 42 | 36 | 40 | 38 | 36 | 42 | 36 | 38 | 60 | 60 | 60 |
| | | 46 | 44 | 38 | 40 | 50 | 45 | 44 | 36 | 40 | 60 | 60 | 60 |
| | | 52 | 60 | 43 | 42 | 60 | 54 | 45 | 36 | 44 | 60 | 60 | 60 |

### Example 15

### Colorimetric Detection of BRD Bacterial Pathogens On-Farm and In-Lab

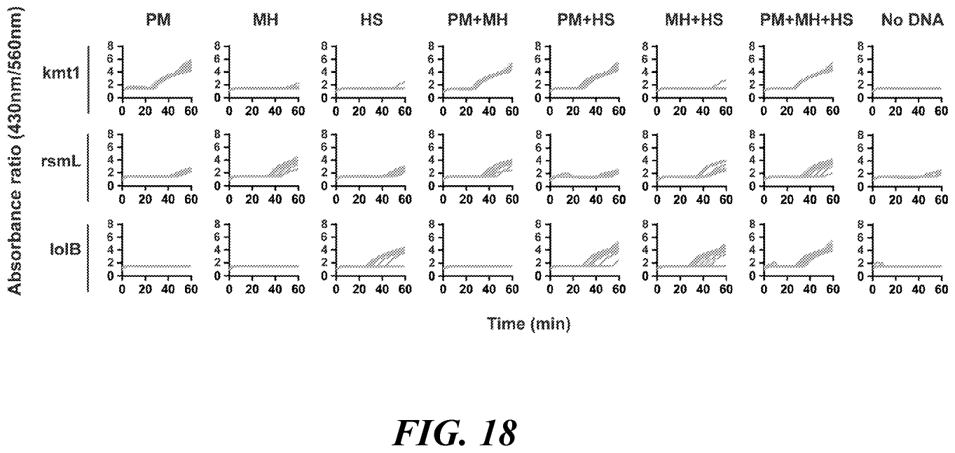

The analytical sensitivity and specificity of the three best-selected primer sets for detecting the identified BRD pathogens identified in Example 12 were evaluated pursuant to the workflow shown in FIG. 11C. The reagents for LAMP reactions were prepared in the lab as described below and mucus from different steers was tested for the presence of P. multocida, M haemolytica, and H. somni both on-farm with a prepared colorimetric and later repeated in-lab. Specifically, LAMP reactions were prepared in individual domed PCR tubes (#AB0337; Thermo Fisher Scientific, Waltham, MA) using 12.5 μL New England Biolabs' Warmstart® Colorimetric LAMP 2× Master Mix, 2.5 μL of primer mix, 5 μL of DNA free water, and 5 μL of mucus sample (in-lab). For the on-farm experiment, the addition of mucus was performed on-farm using a 0.5-10 single-channel pipette with no additional measures to avoid contamination (see FIG. 20). The mucus addition on-farm happened no more than 30 minutes after extraction from the steers, while the mucus addition in the lab was done 4 days after collecting the samples (the samples being stored at −80° C. in the meantime). In both lab and on-farm studies, the swab contents were resuspended and stored in 200 μL water so the test matrix would be similar between the two cohorts.

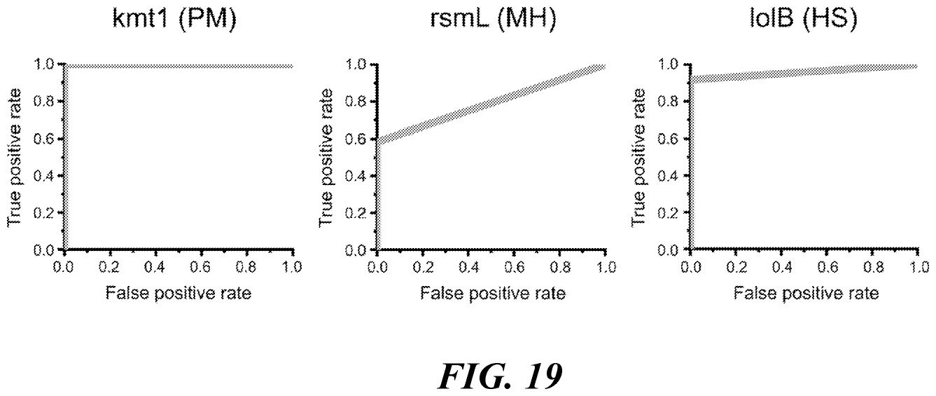

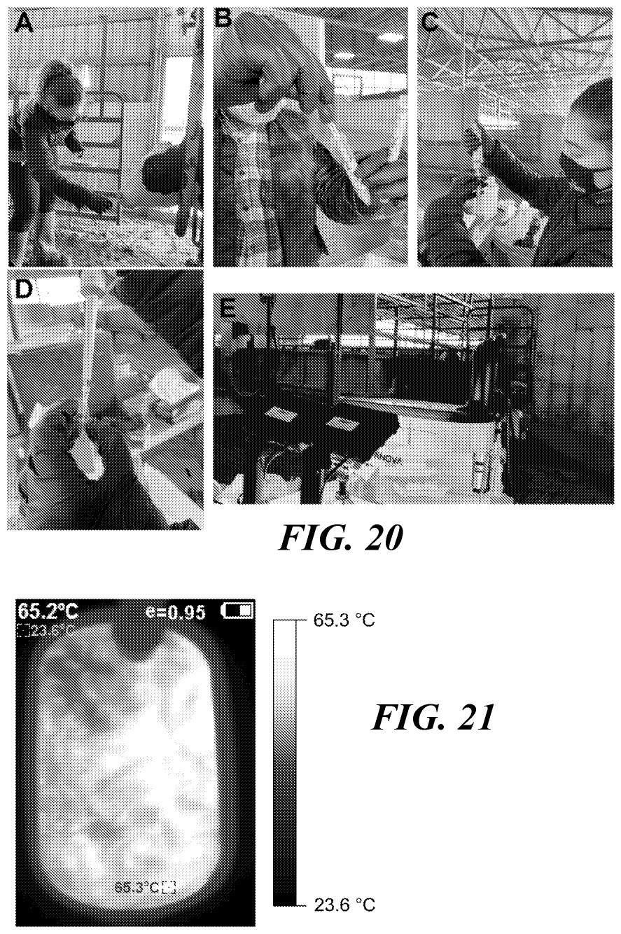

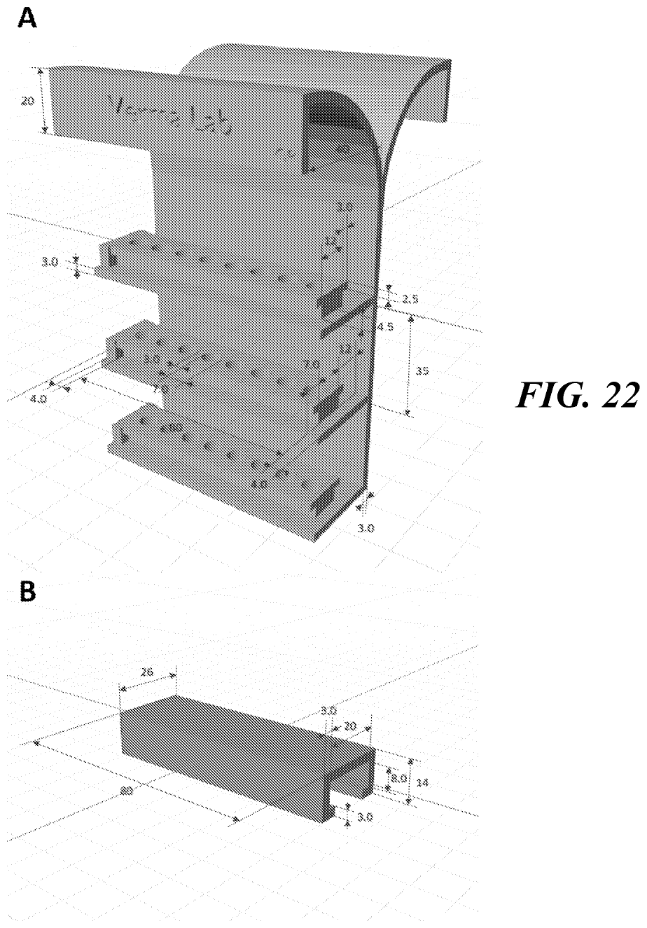

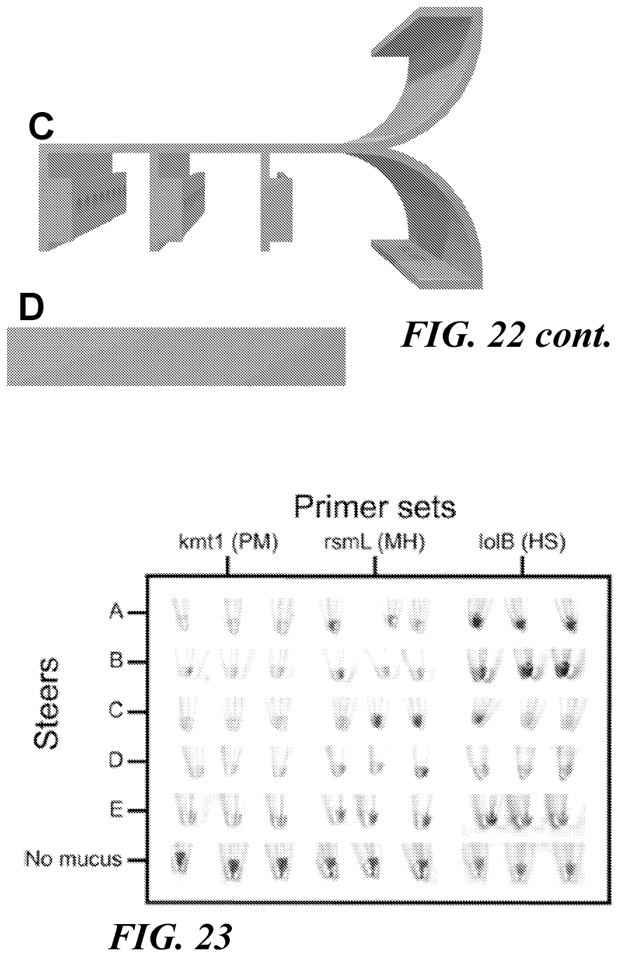

A precision cooker was used as a heating device to confirm the ability to test in a resource-limited setting. Specifically, an Anova Culinary AN500-US00 Sous Vide Precision Cooker (B08CF6Y4WF; Amazon.com, Inc., Seattle, WA) was filled with water and set to 149° F. (65° C.). The temperature of the water was verified in the lab using an Hti HT-04 Thermal Imaging Camera (see FIG. 21). The tubes were submerged in the water on the right side (the region with a relatively homogenous temperature of 65° C.) either by taping them to the inside of the precision cooker with heat-resistant ¾-inch autoclave tape (Thermo Fisher Scientific, Waltham, MA) or by using PCR tube holders designed and 3D-printed in-lab with a Formlabs Form 3B 3D printer (Formlabs, Somerville, MA) using high-temperature resin v2 and 0.1 mm layer thickness (see FIGS. 22A-22D). The tubes were removed from the water after 60 minutes. Images of the tubes in both experimental settings were taken at 0 minutes and 60 minutes (see FIG. 23 (in farm cohort) and FIG. 24 (in-lab cohort)). Images of the tubes in-lab were taken using the Epson Perfection V800 Photo scanner and images of tubes in-farm were taken using a Samsung Galaxy A50. All images obtained were adjusted by using the white balance tool on Adobe Lightroom to obtain a relatively uniform background.

The RGB values of each solution were extracted at 60 minutes using ImageJ and Hue values were calculated to differentiate positive and negative results. Shadows and glows on the images were avoided during this process to increase the accuracy of the results. The Hue scale indicated on a color wheel from 0° to 360°. For reference, red/pink color is around 0-15° and 345-360°, and orange/yellow is around 30-60°. As a Hue value of 35 was set as cut-off (i.e. higher than 35 was considered a positive reaction), the red/pink color on the high end (i.e. close to 360°) was simply set to 0 to avoid confusion.

The concordance observed between LAMP assays performed on the farm and in the lab varied between primers: kmt1 83.3%, rsmL 66.7%, lolB 66.7%. Although lower than expected, in all cases (except one) in which discordance was observed, the results were positive on-farm and negative in-lab. Therefore, the lack of consistency was likely due to the instability of the mucus samples (rather than the unreliability of the LAMP assay). These results further accentuate the necessity of field-based testing.

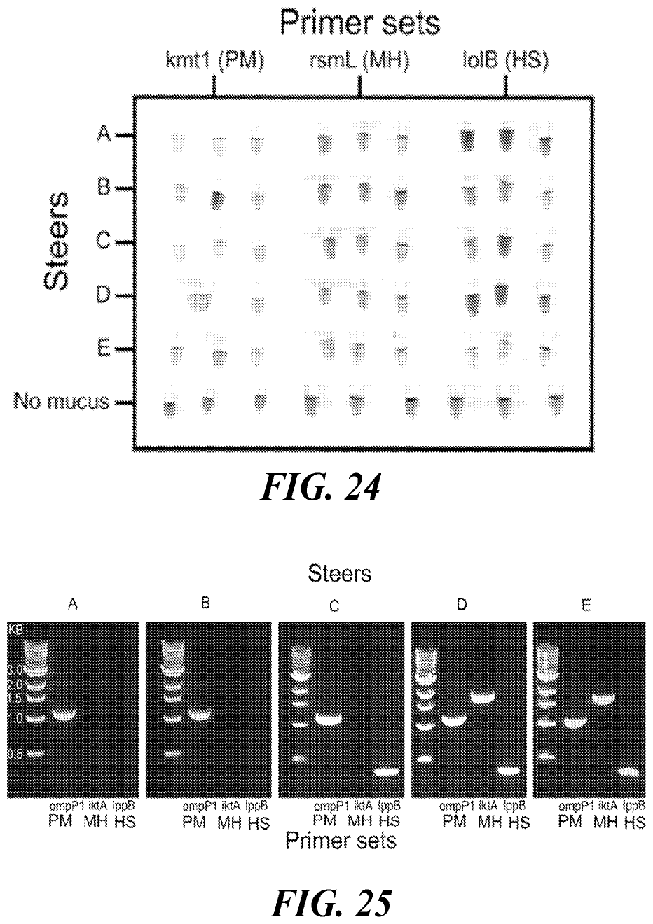

The mucus samples used were also analyzed in a PCR study. Within PCR, P. multocida was detected in all steers, M. haemolytica was only present in steers D and E, and H. somni was detected in steers C, D, and E (see FIG. 25). Of the 5 steers, 5 out of 5 of the ones tested for P. multocida, M haemolytica, and H. somni showed the same results between on-farm LAMP and PCR.

In contrast, only 3 out of 5 of the steers tested for M. haemolytica were in agreement. These results support that LAMP is at least as reliable as PCR in detecting the presence of pathogens as long as optimal primers are designed and employed.

Table 7 shows the percent concordance between in-lab and on-farm LAMP using unprocessed mucus collected from steers.

TABLE 7
Concordance between experiments in-lab and on-farm, and
between the precision cooker assay on-farm and PCR

| | % Concordance: | | | | | | | |
|---|---|---|---|---|---|---|---|---|
| | precision | % Concordance: | | | | | | |
| | cooker | precision cooker | | | | | | |
| Target | on-farm vs. | on-farm vs. | | | | | | |
| pathogen | In-lab PCR (%) | in-lab (%) | | | | | | |
| R. multocida | 100.0 | 83.3 | | | | | | |
| M. haemolytica | 60.0 | 66.7 | | | | | | |
| H. somni | 100.0 | 66.7 | | | | | | |
| Steer | Target pathogen | Farm | | | | | | |
| A | P. multocida | 42 | 40 | 28 | 54 | 48 | 41 | + |
| | M. haemolytica | 5 | 17 | 32 | 10 | 14 | 31 | − |
| | H. somni | 0 | 0 | 0 | 0 | 0 | 0 | − |
| B | P. multocida | 40 | 47 | 47 | 43 | 0 | 34 | + |
| | M. haemolytica | 14 | 38 | 37 | 11 | 0 | 0 | − |
| | H. somni | 29 | 2 | 6 | 20 | 17 | 39 | − |
| C | P. multocida | 39 | 42 | 41 | 54 | 47 | 40 | + |
| | M. haemolytica | 37 | 0 | 0 | 11 | 18 | 16 | − |
| | H. somni | 1 | 35 | 35 | 23 | 0 | 24 | + |
| D | P. multocida | 43 | 46 | 44 | 46 | 34 | 38 | + |
| | M. haemolytica | 33 | 42 | 0 | 17 | 15 | 28 | + |
| | H. somni | 41 | 43 | 40 | 0 | 0 | 0 | + |
| E | P. multocida | 41 | 42 | 31 | 41 | 30 | 29 | + |
| | M. haemolytica | 33 | 31 | 33 | 34 | 32 | 27 | + |
| | H. somni | 21 | 35 | 37 | 48 | 45 | 34 | + |
| Negative | P. multocida | 2 | 0 | 0 | 0 | 0 | 0 | |
| | M. haemolytica | 18 | 21 | 21 | 0 | 0 | 0 | |
| | H. somni | 0 | 0 | 0 | 0 | 0 | 0 | |

A point to note regarding the rsmL primer set for targeting M. haemolytica is that, while the primer set was performing well in the pure M. haemolytica sample, it had around 79% accuracy when used to target M. haemolytica in a mixed sample (mainly due to false negatives). It is thought this reduction in performance is due to cross-reactivity with other off-target DNA.

The below primer screening code was written pursuant to the present disclosure. This code: 1) inputs the raw data from the conducted primer screening experiments described herein, 2) graphs and appropriately labels the data to their corresponding primer set, 3) normalizes and scores the performance for each primer set using the performance information and preassigned weights, 4) determines the best primer set to use for each gene target, and 5) generates a table that displays all primer set performance info and scores that are shown in Table 4.

The code (and functions mentioned below) can be used in MATLAB as long as the mentioned data file (Primer Set Screening 1 Raw Data).

The graphing through this code can be done using a downloaded Matlab figure class (https://www.mathworks.com/matlabcentral/fileexchange/54114-tfigure?s_tid=srchtitle); however, all primer screening graphs displayed herein and code were manually prepared using Origin Pro.

Primer Screening Code (main)

```
clc
clear
close all
[data, text, alldata] = xlsread(′Primer Set Screening 1 Raw Data′);
%% Primer Set names
% Creates vector with all primer set names and removes empty spaces
primer_set_names = string(text);
for i=1:length (primer_set_names)
    if primer_set_names (i) == ″″
        primer_set_index (i) = 1;
    end
end
primer_set_names(find(primer_set_index == 1)) = [ ];
%% Graph Primer Data
%Makes GUI with all primer set graph information present
h = tfigure(′qLAMP Plots′);
j = 1;
%creates and adds individual primer graphs to GUI
for i = 1:16:length(data)
    addPlot(h,convertStringsToChars(primer_set_names(j)));
    primer_set = data (:,i:(i+15));
    primer_plot(primer_set, primer_set_names(j));
    j = j+1;
end
%% Primer Information Extraction
% Extracts primer decision criteria from each primer set screening data
time_avg = [ ];
time_stdev = [ ];
max_avg = [ ];
max_stdev = [ ];
false_pos_total = [ ];
true_neg_false_pos = [ ];
isValid = [ ];
primer_completion = ones(1,27);
%specifies what the target bacteria for each bacteria in primer set
screening is
primer_bacteria_target = [ ones (1,9)*3 ones (1,9)*2 ones (1,9)*4];
j = 1;
for i =1:16:length(data)
    %if a primer set has been completed, extract performance
    %characteristics of primer set and add information to master
performance arrays
    if primer_completion(j) == 1
        primer_set = data(:,i:(i+15));
        [time_avg_temp,max_avg_temp, time_stdev_temp, max_stdev_
temp,false_pos_total_temp, true_neg_false_pos_ temp,
isValid_temp]=primer_info(primer_set, primer_bacteria_target(j));
        time_avg = [time_avg, time_avg_temp];
        time_stdev = [time_stdev, time_stdev_temp];
        max_avg = [max_avg, max_avg_temp];
        max_stdev = [max_stdev, max_stdev_temp];
        false_pos_total = [false_pos_total, false_pos_total_temp];
        true_neg_false_pos = [true_neg_false_pos,
true_neg_false pos_temp];
        isValid = [isValid, isValid_temp];
        j = j+1;
    %else mark all performance characteristics as not completed in master
    %performance arrays
    else
        time_avg = [time_avg, -1];
        time_stdev = [time_stdev, -1];
        max_avg = [max_avg, -1];
        max_stdev = [max_stdev, -1];
        false_pos_total = [false_pos_total, -1];
        true_neg_false_pos = [true_neg_false_pos, -1];
        isValid = [isValid, -1];
        j = j+1;
    end
end
%% Primer Set Selection
%Generates weighted scores for each primer set criteria and picks best
%primer set for each gene
time_avg_score = [ ];
time_stdev_score = [ ];
max_avg_score = [ ];
max_stdev_score = [ ];
false_pos_score = [ ];
total_score = [ ];
[time_avg_score, time_stdev_score, max_avg_score, max_stdev_score,
false_pos_score, total_score] = score2(time_avg, max_avg, time_stdev,
max_stdev, false_pos_total, isValid);
decision_temp = string( );
decision = string( );
%Determines which primer set to use based off of total primer set scores
for i = 1:3:27
    temp = [total_score(i) total_score(i+1) total_score(i+2)];
    true = find(temp == max(temp));
    false = find(temp ~= max(temp));
    decision_temp(true) = ′Use′;
    decision_temp(false) = ′X′;
    decision = [decision, decision_temp];
end
decision(1) = [ ];
%indicates failed primer sets and generates table with all relavent
%performance characteristics, scores, and decisions
yes = find(isValid == 1);
no = find(isValid == 0);
isValidtext = strings ([1,27]);
isValidtext(yes) = ′Yes′;
isValidtext(no) = ′No′;
%isValidtext(primers_not_completed) = ′N/A′;
table_names = {′Reaction_Time_min′, ′Reaction_Time_stdev′,
′Max_Intensity_RFU′, ′Max_Intensity_stdev′, ′Total_False_Positives′,
′True_Neg_False_Positives′, ′Any_Negative_Reactions_pre_30_minutes′
′Total_Score′, ′Decision′};
T = table( time_avg′, time_stdev′, max_avg′, max_stdev′, false_pos_total′,
true_neg_false_pos′, isValidtext′, total_score′, decision′);
T.Properties.VariableNames = table_names;
T.Properties.RowNames = primer_set_names;
```

The below Primer Info Function extracts the performance information from a given primer set based on the inputted raw data series for such primer set and the bacterial target for the primer set.

Primer Info Function

```
function [time_avg, max_avg, time_stdev, max_stdev, false_pos_total,
true_neg_false_pos, isValid] = primer_info(primer_set, positive_bacteria)
%primer_info.m; extracts performance information from a given primer set.
% determines which bacteria of inputted primer set is the target pathogen
if positive bacteria == 1
    num = 1;
elseif positive_bacteria == 2
    num = 5;
elseif positive_bacteria == 3
    num = 9;
elseif positive_bacteria == 4
    num = 13;
end
%references start of fluorescent data for different bacterias in screen
num_series = [ 1 5 9 13];
time = [ ];
max_intensity = [ ];
%determines max intensity, 90% max, and response time for taret pathogen
%replicates
for i = 1:4
    max_intensity(i) = max(primer set(:,num+i-1));
    ninety_percent_max = 0.9*max (primer set (:,num+i-1));
    time(i) = min(find(primer_set(:,num+i-1) >= ninety_percent_max));
end
%averages target replicate performance criteria and determines spread of
%replicate data
max_avg = mean(max_intensity);
time_avg = mean(time);
max_stdev = std(max_intensity);
time_stdev = std(time);
%sets fluorescent intensity threshold for determining false postive
%reactions
false_pos_threshold = 0.2*max_avg;
false_pos_total = 0;
true_neg_false_pos = 0;
%removes target bacteria from bacteria reference array for false positive
%screening
num_series(find(num_series == num))=[ ];
isValid = 0;
for i = num_series
    for j = 1:4
      %if response time of non target reactions is less than 30 minutes,
      %mark the set for rejection
      if min(find(primer_set(:,i+j-1) >= ninety_percent_max)) <=30
          isValid = 1;
      end
          %if non target reactions have amplified past false positive
          %threshold, count them as false positives
      if isempty(find(primer_set(:,i+j-1) > false_pos_threshold)) == 0
          false_pos_total = false_pos_total +1;
      end
      % if Water reactions amplify past false positive
      % threshold, count them as negative control false positives
      if i == 1
          if isempty(find(primer_set(:,i+j-1) > false_pos_threshold)) ==
0
        true_neg_false_pos = true_neg_false_pos + 1;
        end
      end
    end
end
end
```

The below Primer Score Function can normalize and score the performance of a selected primer set upon input of such primer set's performance information (determined earlier in the code).

Primer Score Function

```
function [time_avg_score, time_stdev_score, max_avg_score,
max_stdev_score, false_pos_score, total_score] = score2 (time_avg, max_
avg, time_stdev, max_stdev, false_pos_total, isValid)
%score2.m; Scores performance characteristics of primer sets based off
%normalized characteristic data and assigned weights for desired reaction
%features
%Weights on performance characteristics
time_avg_weight = 20;
time_stdev_weight = 30;
max_avg_weight = 10;
max_stdev_weight = 10;
false_pos_weight = 30;
A = time_avg;
B = time_stdev;
C = max_avg;
D = max_stdev;
E = false_pos_total;
%leaves unfinished primer sets out of score calculation
index = find (A == -1);
A(index) = [ ];
B(index) = [ ];
C(index) = [ ];
D(index) = [ ];
E(index) = [ ];
time_avg_range = range(A);
time_stdev_range = rang (B);
max_avg_range = range(C);
max_stdev_range = range(D);
false_pos_range = range(E);
%calculates score of response time for each primer set
for i=1:length(time_avg)
    if time_avg(i) ~= -1
        time_avg_score(i) = (1-((time_avg(i)-
min(A))/time_avg_range))*time_avg_weight;
    else
        time_avg_score(i) = -1;
    end
end
%calculates score for response time spread for each primer set
for i=1:length(time_stdev)
    if time stdev (i) ~= -1
        time_stdev_score(i)=(1-((time_stdev(i)-
min(B))/time_stdev_range))*time_stdev_weight;
    else
        time_stdev_score(i) = -1;
    end
end
%calculates score for maximum intensity for each primer set
for i=1:length(max_avg)
    if max_avg(i) ~= -1
        max_avg_score(i) = ((max_avg (i)-
min(C))/max_avg_range)*max_avg_weight;
    else
        max_avg_score(i) = -1;
    end
end
%calculates score for max intensity spread for each primer set
for i=1:length(max_stdev)
    if max_stdev(i) ~= -1
        max_stdev_score(i)=(1-((max_stdev(i)-
min(D))/max_stdev_range))*max_stdev_weight;
    else
        max_stdev_score(i) = -1;
    end
end
%calculates score for false positive totals for each primer set
for i=1:length(false_pos_total)
    if false_pos_total(i) ~= -1
        false_pos_score(i)=(1-((false_pos_total(i)-
min(E))/false_pos_range))*false_pos_weight;
    else
        false_pos_score(i) = -1;
    end
end
%calculates total score for every primer set
total_score = time_avg_score + time_stdev_score + max_avg_score +
max_stdev_score + false_pos_score;
%if any primer set had any non target amplification before 30 minutes
%(indicative of cross reactivity), automatically give a failing score to
it
for i = 1:length(isValid)
    if isValid(i) == 1
        total_score (i) = 0;
    end
end
End
```

1. A loop-mediated isothermal amplification (LAMP) assay comprising at least one LAMP primer set that targets a deoxyribonucleic acid (DNA) fragment of a pathogen associated with a bovine respiratory disease (BRD) in a sample, wherein the assay allows for single-step identification of the pathogen.

2. The assay of claim 1, wherein the pathogen associated with BRD is a bacterium, bacterial mycoplasm, fungus, virus, or any other infectious agent associated with BRD.

3. (canceled)

4. The assay of claim 1, wherein the pathogen is selected from the group consisting of Pasteurella multocida (P. multocida), Mannheimia haemolytica (M. haemolytica), and Histophilus somni (H. somni).

5. (canceled)

6. The assay of claim 1, wherein the assay can process and provide a visual result in 60 minutes or less, the visual result indicative of the presence or absence of the pathogen for BRD in the sample.

7.-8. (canceled)

9. The assay of claim 1, wherein the sample is a bovine nasal sample or a bovine water sample, and further comprising a pH- or magnesium-based indicator.

10.-11. (canceled)

12. The assay of claim 1, wherein each of the LAMP primer sets has a limit of detection (LoD) of at least 10³ copies/reaction.

13. The assay of claim 1, wherein:
the pathogen is P. multocida and the targeted DNA fragment comprises a gene selected from the group consisting of kmt1, ompP1, and ompl6;
the pathogen is M. haemolytica and the targeted DNA fragment comprises a gene selected from the group consisting of rsmL, rsmC, and lktA; or
the pathogen is H. somni and the targeted DNA fragment comprises a gene selected from the group consisting of lolA, lolB, and lppB.

14.-16.(canceled)

17. The assay of claim 1, wherein each of the LAMP primer sets is coupled with a colorimetric reagent.

18. The assay of claim 17, wherein the colorimetric reagent is pH sensitive or magnesium sensitive.

19. (canceled)

20. The assay of claim 1, wherein each LAMP primer set comprises one or more primers of SEQ ID NOS. 1-6, 25-30, 55-62, 99-110, and 147-158.

21. The assay of any one of claim 1, wherein each LAMP primer set is at least about 98% specific to the targeted DNA fragment.

22. The assay of claim 1, further comprising a fluorescent indicator.

23. The assay of claim 1, wherein each of the least one LAMP primer set comprises 4 to 6 primers.

24. A method for identification of a pathogen associated with a bovine respiratory disease (BRD) in a sample and treatment thereof comprising:
providing at least one LAMP primer set that targets a deoxyribonucleic acid (DNA) fragment of a targeted pathogen associated with a bovine respiratory disease (BRD) in a sample;
obtaining a sample from a subject;
combining the sample and the at least one LAMP primer set into a mixture;
heating the combination to initiate amplification of the targeted DNA fragment; and
detecting a visual result in the heated combination indicative of the presence or absence of the targeted pathogen for BRD in the sample;
wherein the at least one LAMP primer set is one or more of primer sets A-AAAA in Tables 2 and 3.

25. The method of claim 24, wherein the visual result is provided in 60 minutes or less of initiating the heating step and the sample is a bovine nasal swab.

26. The method of claim 24, wherein detecting a visual result further comprises one or more of:
measuring a relative clarity of the heated combination using a turbidimeter; and
analyzing colorimetric data in the visual result using one or more of a fluorescent reader, an ultraviolet light reader, or camera; and
wherein if the visual result is indicative of the presence of the targeted pathogen, the method further comprises treating the subject for the targeted pathogen.

27.-31. (canceled)

32. A kit comprising:
at least one LAMP primer set that targets a deoxyribonucleic acid (DNA) fragment of a pathogen associated with a bovine respiratory disease (BRD) in a sample;
at least one swab for obtaining the sample; and
a heating element to initiate amplification of the targeted DNA fragment when the at least one LAMP primer set and the sample are combined.

33. The kit of claim 32, further comprising:
a fluorescent indicator; and
a fluorescent reader, an ultraviolet light reader, or a camera to provide colorimetric result data indicative of the presence or absence of a targeted pathogen in the sample.

34. The kit of claim 32, wherein the at least one swab comprises a nasal swab and the kit further comprises a sealable container with a transport media therein.

35. The kit of claim 32, wherein the kit is portable and capable of use in a non-laboratory setting.

36.-38. (canceled)

39. The kit of claim 32, wherein:
the at least one primer set is coupled with a colorimetric reagent that is pH sensitive or magnesium sensitive and the colorimetric reagent is phenol red; and
the at least one LAMP primer set targets a kmt1 fragment of P. multocida or a lolB fragment of H. somni.

40. (canceled)


SEQUENCE LISTING (158 sequences)

SEQ ID NO: 1 - DNA, 17 bp, Artificial Sequence
  LAMP primer kmt1.2 F3
gaatcaagcggtcacag

SEQ ID NO: 2 - DNA, 18 bp, Artificial Sequence
  LAMP primer kmt1.2 B3
cactcacaacgagccata

SEQ ID NO: 3 - DNA, 45 bp, Artificial sequence
  LAMP primer kmt1.2 FIP
agagcagtaatgtcagcacaatattaaagacagcaatttcgagca

SEQ ID NO: 4 - DNA, 40 bp, Artificial Sequence
  LAMP primer kmt1.2 BIP
cgctatttacccagtggggcgccatttcccatttcaagtg

SEQ ID NO: 5 - DNA, 20 bp, Artificial Sequence
  LAMP primer kmt1.2 LF
cgtaaagccccaccattgtt

SEQ ID NO: 6 - DNA, 22 bp, Artificial Sequence
  LAMP primer kmt1.2 LB
accgattgccgcgaaattgagt

SEQ ID NO: 7 - DNA, 21 bp, Artificial Sequence
  LAMP primer ompP1.2 F3
gcaatttatgtggacccaaat

SEQ ID NO: 8 - DNA, 18 bp, Artificial Sequence
  LAMP primer ompP1.2 B3
aatcggttttaccgccta

SEQ ID NO: 9 - DNA, 40 bp, Artificial Sequence
  LAMP primer ompP1.2 FIP
tcggaactaacgcattcggcttaacttcaccaatgccagg

SEQ ID NO: 10 - DNA, 47 bp, Artificial Sequence
  LAMP primer ompP1.2 BIP
atccaattaacgaaaaattcgctgtgcatatttgtcatcaaactcgg

SEQ ID NO: 11 - DNA, 19 bp, Artificial Sequence
  LAMP primer ompP1.2 LF
caatatttttataggcgaa

SEQ ID NO: 12 - DNA, 21 bp, Artificial Sequence
  LAMP primer ompP1.2 LB
ggcggtggattgaatgtcaac

SEQ ID NO: 13 - DNA, 20 bp, Artificial Sequence
  LAMP primer omp16.1 F3
ggcggttattcagtacaaga

SEQ ID NO: 14 - DNA, 18 bp, Artificial Sequence
  LAMP primer omp16.1 B3
catctgcacgacgttgac

SEQ ID NO: 15 - DNA, 48 bp, Artificial Sequence
  LAMP primer omp16.1 FIP
cgcatgtgcatctaaaatttgtacagttataataccgtgtatttcggc

SEQ ID NO: 16 - DNA, 43 bp, Artificial Sequence
  LAMP primer omp16.1 BIP
aatgcaacacctgcaacgaactaatgcgatgttatattctggt

SEQ ID NO: 17 - DNA, 18 bp, Artificial Sequence
  LAMP primer omp16.1 LF
cgatattgtatttatcga

SEQ ID NO: 18 - DNA, 21 bp, Artificial Sequence
  LAMP primer omp16.1 LB
cgttgttgaaggtaacaccga

SEQ ID NO: 19 - DNA, 18 bp, Artificial Sequence
  LAMP primer lktA.3 F3
gtaacgacggcaatgacc

SEQ ID NO: 20 - DNA, 23 bp, Artificial Sequence
  LAMP primer lktA.3 B3
atcttttaagttcgaatcagaga

SEQ ID NO: 21 - DNA, 44 bp, Artificial Sequence
  LAMP primer lktA.3 FIP
ttgcctttaccgccatcgataaagtaaaggcgatgatattctcg

SEQ ID NO: 22 - DNA, 38 bp, Artificial Sequence
  LAMP primer lktA.3 BIP
ggtggcaagggcgatgatatcattgccgtcagaatcgg

SEQ ID NO: 23 - DNA, 18 bp, Artificial Sequence
  LAMP primer lktA.3 LF
tcatcaccatttccacca

SEQ ID NO: 24 - DNA, 20 bp, Artificial Sequence
  LAMP primer lktA.3 LB
tcgttcaccgtaaaggcgat

SEQ ID NO: 25 - DNA, 18 bp, Artificial Sequence
  LAMP primer rsmI.2 F3
cgaagacactcgccacag

SEQ ID NO: 26 - DNA, 19 bp, Artificial Sequence
  LAMP primer rsmI.2 B3
aacttttaccccggcttgg

SEQ ID NO: 27 - DNA, 42 bp, Artificial Sequence
  LAMP primer rsmI.2 FIP
aacgaccgctttctgctgttcattattgctgagccactacgg

SEQ ID NO: 28 - DNA, 40 bp, Artificial Sequence
  LAMP primer rsmI.2 BIP
tgcgttaatttccgatgccggacggcaatgacggacaaga

SEQ ID NO: 29 - DNA, 25 bp, Artificial Sequence
  LAMP primer rsmI.2 LF
gtgcaaggcgaaaaacggtttttta

SEQ ID NO: 30 - DNA, 19 bp, Artificial Sequence
  LAMP primer rsmI.2 LB
gccactgattagcgacccg

SEQ ID NO: 31 - DNA, 18 bp, Artificial Sequence
  LAMP primer rsmC.3 F3
cggcagacgtactttggc

SEQ ID NO: 32 - DNA, 19 bp, Artificial Sequence
  LAMP primer rsmC.3 B3
atgcgtttgcgacaattcg

SEQ ID NO: 33 - DNA, 40 bp, Artificial Sequence
  LAMP primer rsmC.3 FIP
gtggaatggtgggttggagacaagagggggaagtggtagc

SEQ ID NO: 34 - DNA, 38 bp, Artificial Sequence
  LAMP primer rsmC.3 BIP
acggggtcgataccgcctacgctcaccgcctttggtta

SEQ ID NO: 35 - DNA, 25 bp, Artificial Sequence
  LAMP primer rsmC.3 LF
ccgttcattaatgtgagagaacaca

SEQ ID NO: 36 - DNA, 23 bp, Artificial Sequence
  LAMP primer rsmC.3 LB
tggaggagttgattttccaagct

SEQ ID NO: 37 - DNA, 20 bp, Artificial Sequence
  LAMP primer lppB.3 F3
agcacaaaaaatactgagca

SEQ ID NO: 38 - DNA, 23 bp, Artificial Sequence
  LAMP primer lppB.3 B3
agagaaggagattatttggaatg

SEQ ID NO: 39 - DNA, 48 bp, Artificial Sequence
  LAMP primer lppB.3 FIP
tgttgcccatacttctaaggttaaattttgtagcctcagttttcaagc

SEQ ID NO: 40 - DNA, 47 bp, Artificial Sequence
  LAMP primer lppB.3 BIP
accgaataaacaaagctatccgatttttgctgattttgctaatgcgg

SEQ ID NO: 41 - DNA, 18 bp, Artificial Sequence
  LAMP primer lppB.3 LF
tttctctgcttcataacc

SEQ ID NO: 42 - DNA, 24 bp, Artificial Sequence
  LAMP primer lppB.3 LB
cgcactttctttgataactctcgt

SEQ ID NO: 43 - DNA, 21 bp, Artificial Sequence
  LAMP primer lolA.2 F3
agtaatgtaacttgggcaaat

SEQ ID NO: 44 - DNA, 23 bp, Artificial Sequence
  LAMP primer lolA.2 B3
gcaataatttgactttcttgagg

SEQ ID NO: 45 - DNA, 47 bp, Artificial Sequence
  LAMP primer lolA.2 FIP
cacttgttgtgtatagtcagcacttcggttaatgagttacaaaatcg

SEQ ID NO: 46 - DNA, 46 bp, Artificial Sequence
  LAMP primer lolA.2 BIP
atgcacagggaaaaaaaatacagctgtttcattgtccatacgaaat

SEQ ID NO: 47 - DNA, 19 bp, Artificial Sequence
  LAMP primer lolA.2 LF
acacatcaattttatttaa

SEQ ID NO: 48 - DNA, 21 bp, Artificial Sequence
  LAMP primer lolA.2 LB
ggaaaaatacaactcaaacgt

SEQ ID NO: 49 - DNA, 22 bp, Artificial Sequence
  LAMP primer lolB.3 F3
gctacgtgaaatgattggtatc

SEQ ID NO: 50 - DNA, 25 bp, Artificial Sequence
  LAMP primer lolB.3 B3
cttttcagaagaatatctttgggta

SEQ ID NO: 51 - DNA, 50 bp, Artificial Sequence
  LAMP primer lolB.3 FIP
gccgacctgataatctgaattttcatattccattacaacaaatagggaac

SEQ ID NO: 52 - DNA, 46 bp, Artificial Sequence
  LAMP primer lolB.3 BIP
gcaagctttacttattcagttgaggatgctttgatctgttcgatag

SEQ ID NO: 53 - DNA, 18 bp, Artificial Sequence
  LAMP primer lolB.3 LF
ctggttgaccttttagcc

SEQ ID NO: 54 - DNA, 18 bp, Artificial Sequence
  LAMP primer lolB.3 LB
gaagtttggagtgctgac

SEQ ID NO: 55 - DNA, 19 bp, Artificial Sequence
  LAMP primer kmt1.1 LF
cgaccatcggttgcatttc

SEQ ID NO: 56 - DNA, 19 bp, Artificial Sequence
  LAMP primer kmt1.1 LB
tggcattgcatggctatca

SEQ ID NO: 57 - DNA, 21 bp, Artificial Sequence
  LAMP primer kmt1.3 F3
cgctgattaatattgtgctga

SEQ ID NO: 58 - DNA, 18 bp, Artificial Sequence
  LAMP primer kmt1.3 B3
cccaacaaaactgtgctt

SEQ ID NO: 59 - DNA, 41 bp, Artificial Sequence
  LAMP primer kmt1.3 FIP
ttcgcggcaatcggttcattcattactgctctatccgctat

SEQ ID NO: 60 - DNA, 46 bp, Artificial Sequence
  LAMP primer kmt1.3 BIP
tatgccacttgaaatgggaaatggccaaataaaagactaccgacaa

SEQ ID NO: 61 - DNA, 17 bp, Artificial Sequence
  LAMP primer kmt1.3 LF
accgccccactgggtaa

SEQ ID NO: 62 - DNA, 22 bp, Artificial Sequence
  LAMP primer kmt1.3 LB
ttttatggctcgttgtgagtgg

SEQ ID NO: 63 - DNA, 19 bp, Artificial Sequence
  LAMP primer ompP1.1 F3
ttccaactagcagaagtgt

SEQ ID NO: 64 - DNA, 18 bp, Artificial Sequence
  LAMP primer ompP1.1 B3
cctggcattggtgaagtt

SEQ ID NO: 65 - DNA, 39 bp, Artificial Sequence
  LAMP primer ompP1.1 FIP
actgccgcattatctgcaatccaacatccggtttagggc

SEQ ID NO: 66 - DNA, 39 bp, Artificial Sequence
  LAMP primer ompP1.1 BIP
gccacgaacccagcattaatacataaattgccccaacag

SEQ ID NO: 67 - DNA, 19 bp, Artificial Sequence
  LAMP primer ompP1.1 LF
ggcttcccccgcataagca

SEQ ID NO: 68 - DNA, 19 bp, Artificial Sequence
  LAMP primer ompP1.1 LB
cttattgaaacaacctgaa

SEQ ID NO: 69 - DNA, 18 bp, Artificial Sequence
  LAMP primer ompP1.3 F3
gctcttgcacctgcatta

SEQ ID NO: 70 - DNA, 18 bp, Artificial Sequence
  LAMP primer ompP1.3 B3
ggtcttaccgtccgtacc

SEQ ID NO: 71 - DNA, 44 bp, Artificial Sequence
  LAMP primer ompP1.3 FIP
cggtagcggtaaatgtaaagtaccagtcctcaaggtattactgc

SEQ ID NO: 72 - DNA, 48 bp, Artificial Sequence
  LAMP primer ompP1.3 BIP
ttctggctatcataaaatgaccgataactctttaaatttgctccattg

SEQ ID NO: 73 - DNA, 20 bp, Artificial Sequence
  LAMP primer ompP1.3 LF
gggatttctttgccacctgt

SEQ ID NO: 74 - DNA, 20 bp, Artificial Sequence
  LAMP primer ompP1.3 LB
ttttgcaatgcactatagct

SEQ ID NO: 75 - DNA, 22 bp, Artificial Sequence
  LAMP primer omp16.2 F3
cagtacaagatttacaacaacg

SEQ ID NO: 76 - DNA, 23 bp, Artificial Sequence
  LAMP primer omp16.2 B3
aaataatgtttaactgcatctgc

SEQ ID NO: 77 - DNA, 47 bp, Artificial Sequence
  LAMP primer omp16.2 FIP
cgcatgtgcatctaaaatttgtacattataataccgtgtatttcggc

SEQ ID NO: 78 - DNA, 44 bp, Artificial Sequence
  LAMP primer omp16.2 BIP
cgttcttaaatgcaacacctgcgatgttatattctggtgtaccg

SEQ ID NO: 79 - DNA, 21 bp, Artificial Sequence
  LAMP primer omp16.2 LF
ccttcgatattgtatttatcg

SEQ ID NO: 80 - DNA, 23 bp, Artificial Sequence
  LAMP primer omp16.2 LB
tgttgaaggtaacaccgatgaac

SEQ ID NO: 81 - DNA, 20 bp, Artificial Sequence
  LAMP primer omp16.3 F3
gcgttcttaaatgcaacacc

SEQ ID NO: 82 - DNA, 18 bp, Artificial Sequence
  LAMP primer omp16.3 B3
tatgctaacacagcacga

SEQ ID NO: 83 - DNA, 43 bp, Artificial Sequence
  LAMP primer omp16.3 FIP
gacgttgacctaatgcgatgttatgttgttgaaggtaacaccg

SEQ ID NO: 84 - DNA, 42 bp, Artificial Sequence
  LAMP primer omp16.3 BIP
caagctggtcaagtatcaacagtagtaagctgcttcatcgtg

SEQ ID NO: 85 - DNA, 19 bp, Artificial Sequence
  LAMP primer omp16.3 LF
ttctggtgtaccgcgttca

SEQ ID NO: 86 - DNA, 22 bp, Artificial Sequence
  LAMP primer omp16.3 LB
cttacggtgaagagaaacctgc

SEQ ID NO: 87 - DNA, 20 bp, Artificial Sequence
  LAMP primer lktA.1 F3
ttctttaaaaactggggcaa

SEQ ID NO: 88 - DNA, 19 bp, Artificial Sequence
  LAMP primer lktA.1 B3
caatagcggtttgaatcgt

SEQ ID NO: 89 - DNA, 50 bp, Artificial Sequence
  LAMP primer lktA.1 FIP
cgctttgactaaatcctgtaaaccactctatattccccaaaattaccaat

SEQ ID NO: 90 - DNA, 40 bp, Artificial Sequence
  LAMP primer lktA.1 BIP
ccgaagagttggggattgagggcctaaactggtttgagct

SEQ ID NO: 91 - DNA, 17 bp, Artificial Sequence
  LAMP primer lktA.1 LF
taccttgttcagtatca

SEQ ID NO: 92 - DNA, 21 bp, Artificial Sequence
  LAMP primer lktA.1 LB
aagaacgcaataatattgcaa

SEQ ID NO: 93 - DNA, 18 bp, Artificial Sequence
  LAMP primer lktA.2 F3
ccgaagagttggggattg

SEQ ID NO: 94 - DNA, 21 bp, Artificial Sequence
  LAMP primer lktA.2 B3
gcattttgtacaatgctttcg

SEQ ID NO: 95 - DNA, 44 bp, Artificial Sequence
  LAMP primer lktA.2 FIP
cggtttgaatcgtgcctaaactggtacaaagagaagaacgcaat

SEQ ID NO: 96 - DNA, 42 bp, Artificial Sequence
  LAMP primer lktA.2 BIP
ctattggcttaactgagcgtggtggcctgctttagttttctg

SEQ ID NO: 97 - DNA, 18 bp, Artificial Sequence
  LAMP primer lktA.2 LF
tttgagctgttgcaatat

SEQ ID NO: 98 - DNA, 22 bp, Artificial Sequence
  LAMP primer lktA.2 LB
ttgtgttatccgctccacaaat

SEQ ID NO: 99 - DNA, 18 bp, Artificial Sequence
  LAMP primer rsmI.1 F3
atttagtcgccgccgaag

SEQ ID NO: 100 - DNA, 18 bp, Artificial Sequence
  LAMP primer rsmI.1 B3
cccgggtcgctaatcagt

SEQ ID NO: 101 - DNA, 41 bp, Artificial Sequence
  LAMP primer rsmI.1 FIP
tggtcgtgcaaggcgaaaaacacagcggtttattgctgagc

SEQ ID NO: 102 - DNA, 40 bp, Artificial Sequence
  LAMP primer rsmI.1 BIP
atgaacagcagaaagcggtcgtcgttccggcatcggaaat

SEQ ID NO: 103 - DNA, 18 bp, Artificial Sequence
  LAMP primer rsmI.1 LF
gttttttaatgccgtagt

SEQ ID NO: 104 - DNA, 25 bp, Artificial Sequence
  LAMP primer rsmI.1 LB
ttagcaaaaggggaaaacattgcgt

SEQ ID NO: 105 - DNA, 18 bp, Artificial Sequence
  LAMP primer rsmI.3 F3
tttagtcgccgccgaaga

SEQ ID NO: 106 - DNA, 20 bp, Artificial Sequence
  LAMP primer rsmI.3 B3
tgacggacaagatgaaagcc

SEQ ID NO: 107 - DNA, 42 bp, Artificial Sequence
  LAMP primer rsmI.3 FIP
atggtcgtgcaaggcgaaaaacacagcggtttattgctgagc

SEQ ID NO: 108 - DNA, 40 bp, Artificial Sequence
  LAMP primer rsmI.3 BIP
tgaacagcagaaagcggtcgttcgttccggcatcggaaat

SEQ ID NO: 109 - DNA, 18 bp, Artificial Sequence
  LAMP primer rsmI.3 LF
gttttttaatgccgtagt

SEQ ID NO: 110 - DNA, 25 bp, Artificial Sequence
  LAMP primer rsmI.3 LB
attagcaaaaggggaaaacattgcg

SEQ ID NO: 111 - DNA, 19 bp, Artificial Sequence
  LAMP primer rsmC.1 F3
tgcgagatgattttgcccg

SEQ ID NO: 112 - DNA, 20 bp, Artificial Sequence
  LAMP primer rsmC.1 B3
acatttcttgccctgcttgg

SEQ ID NO: 113 - DNA, 39 bp, Artificial Sequence
  LAMP primer rsmC.1 FIP
ctcactatggtggcgtgcgtatcgtgcggaaaaggttgc

SEQ ID NO: 114 - DNA, 41 bp, Artificial Sequence
  LAMP primer rsmC.1 BIP
ttgagtgccgagttagcggtgacagccattgtagcagttgg

SEQ ID NO: 115 - DNA, 20 bp, Artificial Sequence
  rsmC.1 LF
tcaaaatagctactgaatac

SEQ ID NO: 116 - DNA, 19 bp, Artificial Sequence
  rsmC.1 LB
aagaacaagcaggaatgcc

SEQ ID NO: 117 - DNA, 20 bp, Artificial Sequence
  rsmC.2 F3
ggaatgccaattccaactgc

SEQ ID NO: 118 - DNA, 20 bp, Artificial Sequence
  rsmC.2 B3
gcaagcggtaggatttccag

SEQ ID NO: 119 - DNA, 38 bp, Artificial Sequence
  rsmC.2 FIP
caccagctcggttttcgccgcaatggctgtcgcaatgc

SEQ ID NO: 120 - DNA, 22 bp, Artificial Sequence
  rsmC.2 BIP
cgaaccgttcggcaatattgcc

SEQ ID NO: 121 - DNA, 21 bp, Artificial Sequence
  rsmC.2 LF
tcaacatttcttgccctgctt

SEQ ID NO: 122 - DNA, 21 bp, Artificial Sequence
  rsmC.2 LB
aatcgactctgcacgccgttg

SEQ ID NO: 123 - DNA, 22 bp, Artificial Sequence
  lppB.1 F3
cacaaaaaatactgagcaggta

SEQ ID NO: 124 - DNA, 23 bp, Artificial Sequence
  lppB.1 B3
agagaaggagattatttggaatg

SEQ ID NO: 125 - DNA, 42 bp, Artificial Sequence
  lppB.1 FIP
ctgttgcccatacttctaaggttagcctcagttttcaagcga

SEQ ID NO: 126 - DNA, 44 bp, Artificial Sequence
  lppB.1 BIP
taccgaataaacaaagctatccgatgctgattttgctaatgcgg

SEQ ID NO: 127 - DNA, 18 bp, Artificial Sequence
  lppB.1 LF
ttatgaagcagagaaaat

SEQ ID NO: 128 - DNA, 24 bp, Artificial Sequence
  lppB.1 LB
cgcactttctttgataactctcgt

SEQ ID NO: 129 - DNA, 20 bp, Artificial Sequence
  lppB.2 F3
acattggtactggaaagcaa

SEQ ID NO: 130 - DNA, 25 bp, Artificial Sequence
  lppB.2 B3
gcagagaaaattttaaccttagaag

SEQ ID NO: 131 - DNA, 51 bp, Artificial Sequence
  lppB.2 FIP
atcaacgatactcacattgttagctttttctcacagtaaaatgtcaattgc

SEQ ID NO: 132 - DNA, 46 bp, Artificial Sequence
  lppB.2 BIP
gtcactgttttgcgtctaaatagttttcctcgcttgaaaactgagg

SEQ ID NO: 133 - DNA, 16 bp, Artificial Sequence
  lppB.2 LF
gtaattgacgacgtag

SEQ ID NO: 134 - DNA, 18 bp, Artificial Sequence
  lppB.2 LB
aaaaaatactgagcaggt

SEQ ID NO: 135 - DNA, 18 bp, Artificial Sequence
  lolA.1 F3
aagtgactgatgcacagg

SEQ ID NO: 136 - DNA, 19 bp, Artificial Sequence
  lolA.1 B3
caaaacaaagggggtatcg

SEQ ID NO: 137 - DNA, 48 bp, Artificial Sequence
  lolA.1 FIP
aggtgtttcattgtccatacgaaatgaaaaaaaatacagcaaggtagt

SEQ ID NO: 138 - DNA, 48 bp, Artificial Sequence
  lolA.1 BIP
ttgcagatggaaaaacattatggttcttaaggtatcttcaacccaatt

SEQ ID NO: 139 - DNA, 19 bp, Artificial Sequence
  lolA.1 LF
ggacgtttgagttgtattt

SEQ ID NO: 140 - DNA, 20 bp, Artificial Sequence
  lolA.1 LB
cgatccttttgttgagcaag

SEQ ID NO: 141 - DNA, 23 bp, Artificial Sequence
  lolA.3 F3
tcaaacgtcctaatttatttcgt

SEQ ID NO: 142 - DNA, 20 bp, Artificial Sequence
  lolA.3 B3
cgtatcggatttttgctcaa

SEQ ID NO: 143 - DNA, 45 bp, Artificial Sequence
  lolA.3 FIP
acttgctcaacaaaaggatcgtagaatggacaatgaaacacctca

SEQ ID NO: 144 - DNA, 44 bp, Artificial Sequence
  lolA.3 BIP
aacagcaaattgggttgaagatacctgttgccaatgacttggat

SEQ ID NO: 145 - DNA, 21 bp, Artificial Sequence
  lolA.3 LF
tccatctgcaataatttgact

SEQ ID NO: 146 - DNA, 21 bp, Artificial Sequence
  lolA.3 LB
gcgataccccctttgttttgc

SEQ ID NO: 147 - DNA, 19 bp, Artificial Sequence
  lolB.1 F3
catcagctttcagcagttc

SEQ ID NO: 148 - DNA, 20 bp, Artificial Sequence
  lolB.1 B3
aaatgatttatgccgacctg

SEQ ID NO: 149 - DNA, 47 bp, Artificial Sequence
  lolB.1 FIP
gttgatatcttttgttgaacgctgatttcacctttcggtattactgt

SEQ ID NO: 150 - DNA, 46 bp, Artificial Sequence
  lolB.1 BIP
aacttgctacgtgaaatgattggttgaattttcatctggttgacct

SEQ ID NO: 151 - DNA, 16 bp, Artificial Sequence
  lolB.1 LF
ttcctttatggtctga

SEQ ID NO: 152 - DNA, 21 bp, Artificial Sequence
  lolB.1 LB
ctctattccattacaacaaat

SEQ ID NO: 153 - DNA, 18 bp, Artificial Sequence
  lolB.2 F3
aacgtttttccactcgct

SEQ ID NO: 154 - DNA, 20 bp, Artificial Sequence
  lolB.2 B3
catttcacgtagcaagttgt

SEQ ID NO: 155 - DNA, 44 bp, Artificial Sequence
  lolB.2 FIP
tgaactgctgaaagctgatgaaaacttggcaatataacaatccg

SEQ ID NO: 156 - DNA, 47 bp, Artificial Sequence
  lolB.2 BIP
agtttcacctttcggtattactgtttgatatcttttgttgaacgctg

SEQ ID NO: 157 - DNA, 17 bp, Artificial Sequence
  lolB.2 LF
taataaagaataggatt

SEQ ID NO: 158 - DNA, 16 bp, Artificial Sequence
  lolB.2 LB
cagaccataaaggaaa